(19) United States
(12) Patent Application Publication
(10) Pub. No.: US 2025/0326864 A1
(43) Pub. Date: Oct. 23, 2025

(54) MONOCLONAL ANTIBODIES TARGETING CONFORMATION-SPECIFIC EPITOPES OF IMMUNOGLOBULIN LIGHT CHAINS OF THE LAMBDA SUBCLASS

(71) Applicants: PARADOX IMMUNOTHERAPEUTICS INC., Toronto (CA); UNIVERSITY HEALTH NETWORK, Toronto (CA)

(72) Inventors: Yulong SUN, Maple, Ontario (CA); Natalie J. GALANT, Kleinberg, Ontario (CA)

(21) Appl. No.: 18/873,104

(22) PCT Filed: Jun. 12, 2023
(86) PCT No.: PCT/IB2023/000367

§ 371 Date: Dec. 9, 2024

Related U.S. Application Data
(60) Provisional application No. 63/351,533, filed on Jun. 13, 2022.

(51) Int. Cl. C07K 16/42

(52) U.S. Cl. CPC C07K 16/4283; C07K 2317/33; C07K 2317/92

(57) ABSTRACT

Provided herein are anti-human λ light chain antibodies or human λ light chain binding antibody fragments that bind an epitope specific of the human λ light chain. Also provided are polynucleotides and vectors encoding the same, and compositions comprising anti-human λ light chain antibodies or human λ light chain binding antibody fragments. The anti-human λ light chain antibodies or human λ light chain binding antibody fragments are useful for measuring human λ light chain in a biological sample comprising contacting the sample with the anti-human λ light chain antibodies or human λ light chain binding antibody fragments. The anti-human λ light chain antibodies or human λ light chain binding antibody fragments are also useful for reducing λ light chain aggregates.

### CROSS REFERENCE

This application claims the benefit of U.S. Provisional App. No. 63/351,533 filed Jun. 13, 2022, which application is incorporated by reference herein in its entirety.

### BACKGROUND

Light chain amyloidosis (AL amyloidosis) is a life-threatening secondary condition that is associated with the primary spectrum disorder, plasma cell dyscrasia. It is a widely underdiagnosed disease caused by the misfolding of free immunoglobulin light chains (LC) which can deposit as light chain amyloid (AL) in heart, kidney, and nervous system. AL amyloidosis occurs in ˜15% of plasma cell dyscrasias, where a single plasma cell clone undergoes unregulated growth, leading to aberrant production and secretion of a large excess of a monoclonal free immunoglobulin kappa (κ) or lambda (λ) light chain protein into the circulation. This increase in light chain protein concentration can lead to a particular type of aggregation and accumulation known as amyloid deposition. Deposition and infiltration of amyloid in vital organs, such as the heart, kidneys and nervous system results in restrictive cardiomyopathy, nephrotic syndrome, hepatomegaly, peripheral neuropathy, and ultimately death.

AL amyloidosis is the most common type of systemic amyloidosis. With an incidence of 9-14 people per million per year in the Western world. Its prevalence is likely to be underestimated as most patients remain undiagnosed until post-mortem. The Amyloidosis Foundation estimates that more than 35,000-45,000 patients in the USA and EU are suffering from this disease, and there are approximately ˜12,000 and ˜18,600 new AL patients diagnosed in the US and EU each year, respectively. The life expectancy of patients diagnosed with AL amyloidosis depends on extent of organ involvement. For patients with cardiac amyloid deposition (approximately 50%), the prognosis is dismal with a median survival of <6 months from the onset of heart failure symptoms if patients remain untreated.

### SUMMARY

Described herein is an epitope of human lambda (λ) light chain and antibodies that bind human λ epitope. The epitope described herein is advantageous because it allows the production and/or screening of antibodies that bind and promote the clearance of misfolded λ light chains, which are the etiological agent of λ light chain amyloidosis. One obstacle to the treatment of light chain amyloidosis is that different patients produce light chains with different amino acid sequences that are not bound equally well by current anti-light chain antibodies that target variable regions. The antibodies described herein target an epitope in the constant region of λ light chains, and, in certain instances, allow for more consistent clinical results and less patient-to-patient variability in outcome. The universality of binding enables the additional benefit of using the anti-human λ light chain antibodies described herein as a part of a diagnostic test for light chain amyloidosis. Additionally, the anti-human λ light chain antibodies described herein are specific for misfolded light chains and have little reactivity to properly folded light chains, thereby reducing the chances of unwanted immunological side-effects attributable to reduced antibody levels and avoids sequestration of the potential therapeutic by unwanted, off-target binding that would hinder potency. Provided herein are anti-lambda light chain antibodies and compositions comprising anti-lambda light chain antibodies for use in treating or diagnosing lambda light chain amyloidosis.

Provided and described herein are anti-human lambda light chain antibodies or human lambda light chain binding fragments thereof comprising: (a) a heavy chain complementarity determining region 1 (H-CDR1) comprising an amino acid sequence set forth in SEQ ID NO: 3; (b) a heavy chain complementarity determining region 2 (H-CDR2) comprising an amino acid sequence set forth in SEQ ID NO: 4; (c) a heavy chain complementarity determining region 3 (H-CDR3) comprising an amino acid sequence set forth in SEQ ID NO: 5; (d) a light chain complementarity determining region 1 (L-CDR1) comprising an amino acid sequence set forth in SEQ ID NO: 6; (e) a light chain complementarity determining region 2 (L-CDR2) comprising an amino acid sequence set forth in SEQ ID NO: 7; and/or (f) a light chain complementarity determining region 3 (L-CDR3) comprising an amino acid sequence set forth in SEQ ID NO: 8. In some embodiments, the anti-human lambda light chain antibody or human lambda light chain binding fragment comprises: a heavy chain variable region comprising the H-CDR1, H-CDR2, and H-CDR3; and/or a light chain variable region comprising the L-CDR1, L-CDR2, and L-CDR3. In some embodiments:

In certain aspects, the heavy chain variable region comprises an amino acid sequence having at least about 80% identity to SEQ ID NO: 1; and/or the light chain variable region comprises an amino acid sequence having at least about 80% identity to SEQ ID NO: 2.

Also provided and described herein are an anti-human lambda light chain antibody or human lambda light chain binding fragment thereof comprising: (a) a heavy chain complementarity determining region 1 (H-CDR1) comprising an amino acid sequence set forth in SEQ ID NO: 13; (b) a heavy chain complementarity determining region 2 (H-CDR2) comprising an amino acid sequence set forth in SEQ ID NO: 14; (c) a heavy chain complementarity determining region 3 (H-CDR3) comprising an amino acid sequence set forth in SEQ ID NO: 15; (d) a light chain complementarity determining region 1 (L-CDR1) comprising an amino acid sequence set forth in SEQ ID NO: 16; (e) a light chain complementarity determining region 2 (L-CDR2) comprising an amino acid sequence set forth in SEQ ID NO: 17; and/or (f) a light chain complementarity determining region 3 (L-CDR3) comprising an amino acid sequence set forth in SEQ ID NO: 18. In some embodiments, the anti-human lambda light chain antibody or human lambda light chain binding fragment thereof comprises: a heavy chain variable region comprising the H-CDR1, H-CDR2, and H-CDR3; and/or a light chain variable region comprising the L-CDR1, L-CDR2, and L-CDR3. In some embodiments, the heavy chain variable region comprises an amino acid sequence having at least about 80% identity to SEQ ID NO: 11; and/or the light chain variable region comprises an amino acid sequence having at least about 80% identity to SEQ ID NO: 12.

Further Provided and described herein are anti-human lambda light chain antibodies or human lambda light chain binding fragments thereof comprising: (a) a heavy chain complementarity determining region 1 (H-CDR1) comprising an amino acid sequence set forth in SEQ ID NO: 23; (b) a heavy chain complementarity determining region 2 (H-CDR2) comprising an amino acid sequence set forth in SEQ ID NO:24; (c) a heavy chain complementarity determining region 3 (H-CDR3) comprising an amino acid sequence set forth in SEQ ID NO: 25; (d) a light chain complementarity determining region 1 (L-CDR1) comprising an amino acid sequence set forth in SEQ ID NO: 26; (e) a light chain complementarity determining region 2 (L-CDR2) comprising an amino acid sequence set forth in SEQ ID NO: 27; and/or (f) a light chain complementarity determining region 3 (L-CDR3) comprising an amino acid sequence set forth in SEQ ID NO: 28.

In some embodiments, the anti-human lambda light chain antibody or human lambda light chain binding fragment thereof comprises: a heavy chain variable region comprising the H-CDR1, H-CDR2, and H-CDR3; and/or a light chain variable region comprising the L-CDR1, L-CDR2, and L-CDR3. In some embodiments, the heavy chain variable region comprises an amino acid sequence having at least about 80% identity to SEQ ID NO: 21; and/or the light chain variable region comprises an amino acid sequence having at least about 80% identity to SEQ ID NO: 22.

Provided and described herein are anti-human lambda light chain antibodies or human lambda light chain binding fragments thereof comprising: (a) a heavy chain complementarity determining region 1 (H-CDR1) comprising an amino acid sequence set forth in SEQ ID NO: 33; (b) a heavy chain complementarity determining region 2 (H-CDR2) comprising an amino acid sequence set forth in SEQ ID NO: 34; (c) a heavy chain complementarity determining region 3 (H-CDR3) comprising an amino acid sequence set forth in SEQ ID NO: 35; (d) a light chain complementarity determining region 1 (L-CDR1) comprising an amino acid sequence set forth in SEQ ID NO: 36; (e) a light chain complementarity determining region 2 (L-CDR2) comprising an amino acid sequence set forth in SEQ ID NO: 37; and/or (f) a light chain complementarity determining region 3 (L-CDR3) comprising an amino acid sequence set forth in SEQ ID NO: 38. In some embodiments, the anti-human lambda light chain antibody or human lambda light chain binding fragment thereof comprises: a heavy chain variable region comprising the H-CDR1, H-CDR2, and H-CDR3; and/or a light chain variable region comprising the L-CDR1, L-CDR2, and L-CDR3. In some embodiments, the heavy chain variable region comprises an amino acid sequence having at least about 80% identity to SEQ ID NO: 31; and/or the light chain variable region comprises an amino acid sequence having at least about 80% identity to SEQ ID NO: 32.

Also provided and described herein are anti-human lambda light chain antibody or human lambda light chain binding antibody fragment thereof comprising: (a) a heavy chain complementarity determining region 1 (H-CDR1) comprising an amino acid sequence set forth in SEQ ID NO: 43; (b) a heavy chain complementarity determining region 2 (H-CDR2) comprising an amino acid sequence set forth in SEQ ID NO:44; (c) a heavy chain complementarity determining region 3 (H-CDR3) comprising an amino acid sequence set forth in SEQ ID NO: 45; (d) a light chain complementarity determining region 1 (L-CDR1) comprising an amino acid sequence set forth in SEQ ID NO: 46; (e) a light chain complementarity determining region 2 (L-CDR2) comprising an amino acid sequence set forth in SEQ ID NO: 47; and/or (f) a light chain complementarity determining region 3 (L-CDR3) comprising an amino acid sequence set forth in SEQ ID NO: 48. In some embodiments, the anti-human lambda light chain antibody or human lambda light chain binding fragment comprises: a heavy chain variable region comprising the H-CDR1, H-CDR2, and H-CDR3; and/or a light chain variable region comprising the L-CDR1, L-CDR2, and L-CDR3. In some embodiments, the heavy chain variable region comprises an amino acid sequence having at least about 80% identity to SEQ ID NO: 41; and/or the light chain variable region comprises an amino acid sequence having at least about 80% identity to SEQ ID NO: 42.

Provided and described herein are anti-human lambda light chain antibody or human lambda light chain antibody fragment thereof comprising: a heavy chain variable region comprising an amino acid sequence having at least about 90%, 95%, 97%, 98%, or 99% identity to SEQ ID NO: 1; and/or a light chain variable region comprising an amino acid sequence having at least about 90%, 95%, 97%, 98%, or 99% identity to SEQ ID NO: 2. Also provided are anti-human lambda light chain antibody or human lambda light chain binding antibody fragment thereof comprising: a heavy chain variable region comprising an amino acid sequence as set forth in SEQ ID NO: 1; and/or a light chain variable region comprising an amino acid sequence as set forth in SEQ ID NO: 2.

Provided and described herein are anti-human lambda light chain antibodies or human lambda light chain binding fragments thereof comprising: a heavy chain variable region comprising an amino acid sequence having at least about 90%, 95%, 97%, 98%, or 99% identity to SEQ ID NO: 11; and/or a light chain variable region comprising an amino acid sequence having at least about 90%, 95%, 97%, 98%, or 99% identity to SEQ ID NO: 12. Also Provided and described herein are anti-human lambda light chain antibodies or human lambda light chain binding fragments thereof comprising: a heavy chain variable region comprising an amino acid sequence as set forth in SEQ ID NO: 11; and/or a light chain variable region comprising an amino acid sequence as set forth in SEQ ID NO: 12.

Provided and described herein are anti-human lambda light chain antibodies or human lambda light chain binding fragments thereof comprising: a heavy chain variable region comprising an amino acid sequence having at least about 90%, 95%, 97%, 98%, or 99% identity to SEQ ID NO: 21; and/or a light chain variable region comprising an amino acid sequence having at least about 90%, 95%, 97%, 98%, or 99% identity to SEQ ID NO: 22. Also Provided and described herein are anti-human lambda light chain antibodies or human lambda light chain binding fragments thereof comprising: a heavy chain variable region comprising an amino acid sequence as set forth in SEQ ID NO: 21; and/or a light chain variable region comprising an amino acid sequence as set forth in SEQ ID NO: 22.

Provided and described herein are anti-human lambda light chain antibodies or human lambda light chain binding fragments thereof comprising: a heavy chain variable region comprising an amino acid sequence having at least about 90%, 95%, 97%, 98%, or 99% identity to SEQ ID NO: 31; and/or a light chain variable region comprising an amino acid sequence having at least about 90%, 95%, 97%, 98%, or 99% identity to SEQ ID NO: 32. Also Provided and described herein are anti-human lambda light chain antibodies or human lambda light chain binding fragments thereof comprising: a heavy chain variable region comprising an amino acid sequence as set forth in SEQ ID NO: 31; and/or a light chain variable region comprising an amino acid sequence as set forth in SEQ ID NO: 32.

Provided and described herein are anti-human lambda light chain antibodies or human lambda light chain binding fragments thereof comprising: a heavy chain variable region comprising an amino acid sequence having at least about 90%, 95%, 97%, 98%, or 99% identity to SEQ ID NO: 41; and/or a light chain variable region comprising an amino acid sequence having at least about 90%, 95%, 97%, 98%, or 99% identity to SEQ ID NO: 42. Also Provided and described herein are anti-human lambda light chain antibodies or human lambda light chain binding fragments thereof comprising: a heavy chain variable region comprising an amino acid sequence as set forth in SEQ ID NO: 41; and/or a light chain variable region comprising an amino acid sequence as set forth in SEQ ID NO: 42.

In some embodiments, the anti-human λ light chain antibody is a monoclonal antibody. In some embodiments, the anti-human λ light chain antibody is a chimeric antibody, a humanized antibody, or human antibody. In some embodiments, the human λ light chain binding antibody fragment comprises a Fab, a Fab′, a F(ab′)₂, a scFv, a dsFv, a ds-scFv, a dimer, a minibody, or a diabody. In some embodiments, the anti-human lambda light chain antibody or human lambda light chain binding fragment thereof is an IgG antibody. In some embodiments, the IgG antibody is an IgG1 isotype. In some embodiments, the IgG antibody is an IgG4 isotype. In some embodiments, the anti-human lambda light chain antibody or human lambda light chain binding fragment thereof binds to an epitope comprising SEQ ID NO: 51. In some embodiments, the binding to the epitope comprising SEQ ID NO: 51 is measured by ELISA, surface plasmon resonance, bio-layer interferometry, or isothermal calorimetry.

In some embodiments, the anti-human lambda light chain antibody or human lambda light chain binding fragment thereof is coupled to a label. In some embodiments, the label comprises a chemical label, a biological label, a fluorescent label, a radioisotope label, or any combination thereof.

Provided and described herein are pharmaceutical compositions comprising the anti-human lambda light chain antibody or human lambda light chain binding fragment thereof, and a pharmaceutically acceptable excipient, diluent, or carrier. Also provided are nucleic acids encoding an anti-human lambda light chain antibody or human lambda light chain binding fragment thereof. Further provided are host cells comprising a nucleic acid encoding the anti-human lambda light chain antibody or human lambda light chain binding fragment.

Provided and described herein are methods of diagnosing amyloidosis in a sample, the method comprising contacting a biological sample obtained from a subject with the anti-human lambda light chain antibodies or human lambda light chain binding fragments thereof described herein. Also provided are methods of detecting free light chain polypeptides in a sample, the method comprising contacting the biological sample with the anti-human lambda light chain antibodies or human lambda light chain binding fragments thereof described herein. Further provided herein are methods of detecting amyloid deposits in a sample, the method comprising contacting the biological sample with the anti-human lambda light chain antibodies or human lambda light chain binding fragments thereof described herein.

In some embodiments, the sample is from blood, serum, plasma, solid tissue, and any combination thereof. In some embodiments, the sample is from a subject.

Provided and described herein are methods of promoting the clearance of a λ light chain aggregate by monocytic cells, the method comprising contacting the λ light chain aggregate with the anti-human lambda light chain antibodies or human lambda light chain binding fragments thereof described herein. Also provided and described herein are methods of increasing the clearance of λ light chain aggregates, the method comprising contacting the λ light chain aggregate with the anti-human lambda light chain antibodies or human lambda light chain binding fragments thereof described herein.

Provided are methods of reducing lambda light chain aggregates in a subject, the method comprising administering the anti-human lambda light chain antibodies or human lambda light chain binding fragments thereof described herein to the subject, thereby reducing the number of lambda light chain aggregates in the subject.

Also provided and described herein are methods of treating a disease or condition characterized by lambda light chain aggregates, the method comprising administering the anti-human lambda light chain antibodies or human lambda light chain binding fragments thereof described herein to the subject, thereby reducing the number of lambda light chain aggregates in the subject.

In some embodiments, the subject is afflicted with a plasma cell disorder. In some embodiments, the plasma cell disorder comprises plasma cell dyscrasias. In some embodiments, the plasma cell dyscrasia is selected from a monoclonal gammopathy of unknown significance (MGUS), multiple myeloma (MM), plasma cell leukemia (PCL), and any combination thereof. In some embodiments, the subject is afflicted with systemic amyloidosis. In some embodiments, the subject is afflicted with AL amyloidosis.

### BRIEF DESCRIPTION OF THE DRAWINGS

The novel features of the present within a composition disclosure are set forth with particularity in the appended claims. A better understanding of the features and advantages of the present within a composition disclosure will be obtained by reference to the following detailed description that sets forth illustrative embodiments, in which the principles of the invention are utilized, and the accompanying drawings of which:

FIG. 1 shows detection of unfolded full-length λ light chain protein by denaturing western blot following denaturing SDS-PAGE, probed using five murine monoclonal antibodies described herein. Samples loaded include kappa (κ) and lambda (λ) light chains pooled from human myeloma sera, as well as λ light chain amyloidosis patient serum (pt). Two of the 5 clones showed reactivity to both λ and a sera.

FIGS. 2A, 2B, 2C, 2D, 2E, and 2F are line graphs illustrating the results of indirect ELISA using five murine monoclonal antibodies described herein (FIGS. 2A-2E) or commercial control antibody (‘Com’, FIG. 2F) to detect native or misfolded forms of λ light chain (Wil). Varying concentrations of native or misfolded Wil were bound and detected using appropriate HRP-conjugated secondary antibodies using TMB chemistry.

FIG. 3 shows images of native Western blot of misfolded, native, or amyloid species probed using 5 murine monoclonal antibodies. Western blots were prepared by transferring from a native PAGE and detecting using appropriate secondary antibodies. Monomeric species of native (n) or misfolded (m) Wil appear as ˜25 kDa bands, while aggregates (a) or large MW species (>250 kDa) appear as a smear in the amyloid (a) sample lane.

FIGS. 4A and 4B, are a graphical representation of results of capture ELISA using misfolded (half-filled circle) or native (full circle) Wil as targets for monoclonal antibody binding. For both clones, increasing concentrations of native Wil resulted in no significant signal change while the same increase in concentration of misfolded Wil resulted in increased binding. These binding curves correspond to affinities of 69 nM (clone 1) and 32 nM (clone 2).

FIGS. 5A and 5B are bio-layer interferometry sensorgrams of binding interactions between Clone 1 (FIG. 5A) and clone 2 (FIG. 5B) to misfolded lambda light chain Wil. Binding of clone 1 and clone 2 with its misfolded Wil at a range of target concentrations (1000 nM to 4 nM) was measured by changes in wavelength (nm) over 500 seconds (120 s association, 300 s dissociation). Association and dissociation curves were fit using Octet Systems Software to determine K_{D}.

FIGS. 6A-6F are bio-layer interferometry sensorgrams of binding interactions between clone 1 (muLX-97) to misfolded (FIGS. 6A, 6B, 6C) and native (FIGS. 6D, 6E, 6F) conformations of Wil (FIGS. 6A, 6D), H3 (FIGS. 6B, 6E), and FOR102 (FIGS. 6C, 6F) lambda light chain variants. Binding of muLX-97 with its misfolded Wil at a range of target concentrations was measured by changes in wavelength (nm) over 720 seconds (120 s association, 600 s dissociation). Association and dissociation curves were fit using Octet Systems Software to determine K_{D}.

FIGS. 7A and 7B are line graphs illustrating the results of binding of misfolded (FIG. 7A) or amyloid (FIG. 7B) Wil to muLX-97 measured by MST (microscale thermophoresis). The shifts in normalized relative fluorescence (Fnorm) during thermophoresis as a function of ligand concentration shows a transition corresponding to a K_{D} of 61.9 nM for misfolded Wil and 87 nM for amyloid Wil.

FIG. 8A-8D are images of immunohistochemistry staining and fluorescence images of biopsy-confirmed cases of λ AL amyloidosis. A gastrointestinal (FIGS. 8A, 8C) and soft tissue case (FIGS. 8B, 8D) were stained (dark regions) using muLX-97 (FIGS. 8A, 8B). The same tissue was co-stained using Congo red amyloid specific dye (FIGS. 8C, 8D) and the fluorescence signal was visualized (white regions). Scale bar=100 μm.

FIGS. 9A-9G are images of immunohistochemistry staining of biopsy-confirmed cases of λAL amyloidosis or pathological controls stained using muLX-97. Gastrointestinal (FIGS. 9A, 9B), Renal (FIG. 9C), and soft tissue (FIGS. 9D, 9E) cases of λ AL amyloidosis stained positive (dark region), but pathological controls of an IAPP amyloid pancreatic tissue (FIG. 9F) and ATTR cardiac amyloid tissue (FIG. 9G) stained negative. Scale bar=100 μm.

FIGS. 10A and 10B are fluorescence images of RAW 264.7 cells in representative fields of view. Cells were incubated with pHrodo-labeled Wil for 30 minutes in the presence of varying concentrations of an isotype control antibody (FIG. 10A) or muLX-97 (FIG. 10B). The pHrodo fluorescence signal (white) represents cells that have internalized Wil amyloid via phagocytic uptake. Scale bar=100 μm.

FIGS. 11A and 11B are fluorescence images of J774.1 cells in representative fields of view. Cells were incubated with pHrodo-labeled Wil for 30 minutes in the presence of varying concentrations of an isotype control antibody (FIG. 11A) or muLX-97 (FIG. 11B). The pHrodo fluorescence signal (white) represents cells that have internalized Wil amyloid via phagocytic uptake. Scale bar=100 μm.

FIGS. 12A and 12B are line graphs showing dose dependent phagocytic uptake of pHrodo labeled amyloid Wil induced by muLX-97 in mouse macrophage cells. 40,000 RAW 264.7 (FIG. 12A) or J774.1 (FIG. 12B) cells were incubated with pHrodo-labeled Wil and either an isotype antibody (circle) or muLX-97 (triangle) at increasing antibody concentrations. Cells with pHrodo fluorescence were imaged (white) and quantified using Biotek Cytation 5 imager. A total cell count was performed using the nuclear stain Nuc Blue live cell reagent to determine % pHrodo positivity. Plotting % pHrodo positive cells as a function of muLX-97 concentration shows a transition corresponding to an EC₅₀ of 20 nM in RAW 264.7 cells and 7 nM in J774.1 cells.

### DETAILED DESCRIPTION

Current treatments for AL amyloidosis are therapies designed to treat the underlying plasma cell dyscrasia (i.e., multiple myeloma) by targeting the cells that produce the toxic light chains. The only on-label therapy currently available is the monoclonal antibody-based therapy daratumumab and hyaluronidase-fihj in combination with bortezomib, cyclophosphamide and dexamethasone. This antibody (and other off-label therapies) targets the CD38 receptor on plasma cells to induce antibody-dependent cellular cytotoxicity. The drug's initial indication is multiple myeloma, but it was approved as an on-label treatment for newly diagnosed AL amyloidosis patients in January 2021. Other treatments for AL amyloidosis include more invasive interventions such as autologous stem cell transplants (ASTC) or standard cytotoxic chemotherapies. However, all the preceding therapies work by targeting the proliferation of the disease-causing plasma cell clone and do not address the already-present amyloid deposits directly affecting organs. While effective for early-stage patients, daratumumab is also not recommended for patients with late-stage cardiac disease. Thus, a non-invasive therapy that does not deplete plasma cell populations to address the existing toxic amyloid deposits to improve organ function of late-stage patients remains a significant challenge and unmet need in treating AL amyloidosis and/or disorders characterized by misfolded antibody molecule.

### Anti-Human λ Light Chain Antibodies

Provided herein are antibodies and antibody fragments that bind human λ light chain. The anti-human λ light chain antibodies described herein are useful and advantageous in that the antibodies and antibody fragments specifically bind to misfolded λ light chains (i.e., as compared to folded λ light chains). Generally, their useful and advantageous can be attributed to the ability of the anti-human λ light chain antibodies to specifically bind to an epitope specific to misfolded λ light chains, SEQ ID NO: 51. The antibodies described provided herein are anti-human λ light chain antibodies that, in certain instances, promote the clearance of misfolded λ light chains (e.g., present in AL amyloidosis or a plasma cell disorder). In some embodiments, the anti-human λ light chain antibodies are useful in methods for reducing amyloid deposits and/or treating diseases characterized by amyloid deposits (e.g., present in AL amyloidosis or a plasma cell disorder). In other instances, the anti-human λ light chain antibodies can be used to diagnose, detect, or screen for misfolded λ light chains, amyloid deposits, and/or a disorder associated therewith.

### Anti-Human λ Light Chain CDRs

Provided and described herein are anti-human λ light chain antibodies and/or human λ light chain binding antibody fragments comprising:
(a) a heavy chain complementarity determining region 1 (H-CDR1) comprising an amino acid sequence set forth in SEQ ID NO: 3;
(b) a heavy chain complementarity determining region 2 (H-CDR2) comprising an amino acid sequence set forth in SEQ ID NO: 4;
(c) a heavy chain complementarity determining region 3 (H-CDR3) comprising an amino acid sequence set forth in SEQ ID NO: 5;
(d) a light chain complementarity determining region 1 (L-CDR1) comprising an amino acid sequence set forth in SEQ ID NO: 6;
(e) a light chain complementarity determining region 2 (L-CDR2) comprising an amino acid sequence set forth in SEQ ID NO: 7; and/or
(f) a light chain complementarity determining region 3 (L-CDR3) comprising an amino acid sequence set forth in SEQ ID NO: 8.
   In some embodiments, the anti-human λ light chain antibodies and/or human λ light chain binding antibody fragments comprise: a heavy chain variable region comprising an amino acid sequence having at least about 70%, at least about 80%, at least about 85%, at least about 90%, at least about 95%, or at least about 98% identity to SEQ ID NO: 1; and/or a light chain variable region comprises an amino acid sequence having at least about 70%, at least about 80%, at least about 85%, at least about 90%, at least about 95%, or at least about 98% identity to SEQ ID NO: 2. In some embodiments, the anti-human λ light chain antibodies and/or human λ light chain binding antibody fragments bind an epitope comprising SEQ ID NO: 51.

Also provided and described herein are anti-human λ light chain antibodies and/or human λ light chain binding antibody fragments comprising:
(a) a heavy chain complementarity determining region 1 (H-CDR1) comprising an amino acid sequence set forth in SEQ ID NO: 13;
(b) a heavy chain complementarity determining region 2 (H-CDR2) comprising an amino acid sequence set forth in SEQ ID NO: 14;
(c) a heavy chain complementarity determining region 3 (H-CDR3) comprising an amino acid sequence set forth in SEQ ID NO: 15;
(d) a light chain complementarity determining region 1 (L-CDR1) comprising an amino acid sequence set forth in SEQ ID NO: 16;
(e) a light chain complementarity determining region 2 (L-CDR2) comprising an amino acid sequence set forth in SEQ ID NO: 17; and/or
(f) a light chain complementarity determining region 3 (L-CDR3) comprising an amino acid sequence set forth in SEQ ID NO: 18.

In some embodiments, the anti-human λ light chain antibodies and/or human λ light chain binding antibody fragments comprise: a heavy chain variable region comprising an amino acid sequence having at least about 70%, at least about 80%, at least about 85%, at least about 90%, at least about 95%, or at least about 98% identity to SEQ ID NO: 11; and/or a light chain variable region comprises an amino acid sequence having at least about 70%, at least about 80%, at least about 85%, at least about 90%, at least about 95%, or at least about 98% identity to SEQ ID NO: 12. In some embodiments, the anti-human λ light chain antibodies and/or human λ light chain binding antibody fragments bind an epitope comprising SEQ ID NO: 51.

Further provided and described herein are anti-human λ light chain antibodies and/or human λ light chain binding antibody fragments comprising:
(a) a heavy chain complementarity determining region 1 (H-CDR1) comprising an amino acid sequence set forth in SEQ ID NO: 23;
(b) a heavy chain complementarity determining region 2 (H-CDR2) comprising an amino acid sequence set forth in SEQ ID NO: 24;
(c) a heavy chain complementarity determining region 3 (H-CDR3) comprising an amino acid sequence set forth in SEQ ID NO: 25;
(d) a light chain complementarity determining region 1 (L-CDR1) comprising an amino acid sequence set forth in SEQ ID NO: 26;
(e) a light chain complementarity determining region 2 (L-CDR2) comprising an amino acid sequence set forth in SEQ ID NO: 27; and/or
(f) a light chain complementarity determining region 3 (L-CDR3) comprising an amino acid sequence set forth in SEQ ID NO: 28.
   In some embodiments, the anti-human λ light chain antibodies and/or human λ light chain binding antibody fragments comprise: a heavy chain variable region comprising an amino acid sequence having at least about 70%, at least about 80%, at least about 85%, at least about 90%, at least about 95%, or at least about 98% identity to SEQ ID NO: 21; and/or a light chain variable region comprises an amino acid sequence having at least about 70%, at least about 80%, at least about 85%, at least about 90%, at least about 95%, or at least about 98% identity to SEQ ID NO: 22. In some embodiments, the anti-human λ light chain antibodies and/or human λ light chain binding antibody fragments bind an epitope comprising SEQ ID NO: 51.

Provided and described herein are anti-human λ light chain antibodies and/or human λ light chain binding antibody fragments comprising:
(a) a heavy chain complementarity determining region 1 (H-CDR1) comprising an amino acid sequence set forth in SEQ ID NO: 33;
(b) a heavy chain complementarity determining region 2 (H-CDR2) comprising an amino acid sequence set forth in SEQ ID NO: 34;
(c) a heavy chain complementarity determining region 3 (H-CDR3) comprising an amino acid sequence set forth in SEQ ID NO: 35;
(d) a light chain complementarity determining region 1 (L-CDR1) comprising an amino acid sequence set forth in SEQ ID NO: 36;
(e) a light chain complementarity determining region 2 (L-CDR2) comprising an amino acid sequence set forth in SEQ ID NO: 37; and/or
(f) a light chain complementarity determining region 3 (L-CDR3) comprising an amino acid sequence set forth in SEQ ID NO: 38.
   In some embodiments, the anti-human λ light chain antibodies and/or human λ light chain binding antibody fragments comprise: a heavy chain variable region comprising an amino acid sequence having at least about 70%, at least about 80%, at least about 85%, at least about 90%, at least about 95%, or at least about 98% identity to SEQ ID NO: 31; and/or a light chain variable region comprises an amino acid sequence having at least about 70%, at least about 80%, at least about 85%, at least about 90%, at least about 95%, or at least about 98% identity to SEQ ID NO: 32. In some embodiments, the anti-human λ light chain antibodies and/or human λ light chain binding antibody fragments bind an epitope comprising SEQ ID NO: 51.

Further provided and described herein are anti-human λ light chain antibodies and/or human λ light chain binding antibody fragments comprising:
(a) a heavy chain complementarity determining region 1 (H-CDR1) comprising an amino acid sequence set forth in SEQ ID NO: 43;
(b) a heavy chain complementarity determining region 2 (H-CDR2) comprising an amino acid sequence set forth in SEQ ID NO: 44;
(c) a heavy chain complementarity determining region 3 (H-CDR3) comprising an amino acid sequence set forth in SEQ ID NO: 45;
(d) a light chain complementarity determining region 1 (L-CDR1) comprising an amino acid sequence set forth in SEQ ID NO: 46;
(e) a light chain complementarity determining region 2 (L-CDR2) comprising an amino acid sequence set forth in SEQ ID NO: 47; and/or
(f) a light chain complementarity determining region 3 (L-CDR3) comprising an amino acid sequence set forth in SEQ ID NO: 48.
   In some embodiments, the anti-human λ light chain antibodies and/or human λ light chain binding antibody fragments comprise: a heavy chain variable region comprising an amino acid sequence having at least about 70%, at least about 80%, at least about 85%, at least about 90%, at least about 95%, or at least about 98% identity to SEQ ID NO: 41; and/or a light chain variable region comprises an amino acid sequence having at least about 70%, at least about 80%, at least about 85%, at least about 90%, at least about 95%, or at least about 98% identity to SEQ ID NO: 42. In some embodiments, the anti-human λ light chain antibodies and/or human λ light chain binding antibody fragments bind an epitope comprising SEQ ID NO: 51.

Further provided and described herein are anti-human λ light chain antibodies and/or human λ light chain binding antibody fragments comprising:
(a) a heavy chain complementarity determining region 1 (H-CDR1) comprising an amino acid sequence set forth in SEQ ID NO: 33 or 43;
(b) a heavy chain complementarity determining region 2 (H-CDR2) comprising an amino acid sequence set forth in SEQ ID NO: 34 or 44;
(c) a heavy chain complementarity determining region 3 (H-CDR3) comprising an amino acid sequence set forth in SEQ ID NO: 35 or 45;
(d) a light chain complementarity determining region 1 (L-CDR1) comprising an amino acid sequence set forth in SEQ ID NO: 36 or 46;
(e) a light chain complementarity determining region 2 (L-CDR2) comprising an amino acid sequence set forth in SEQ ID NO: 37 or 47; and/or
(f) a light chain complementarity determining region 3 (L-CDR3) comprising an amino acid sequence set forth in SEQ ID NO: 38 or 48;
   wherein the anti-human λ light chain antibodies and/or human λ light chain binding antibody fragments bind an epitope comprising SEQ ID NO: 51. In some embodiments, the anti-human λ light chain antibodies and/or human λ light chain binding antibody fragments comprise: a heavy chain variable region comprising an amino acid sequence having at least about 70%, at least about 80%, at least about 85%, at least about 90%, at least about 95%, or at least about 98% identity to SEQ ID NO: 31 or 41; and/or a light chain variable region comprises an amino acid sequence having at least about 70%, at least about 80%, at least about 85%, at least about 90%, at least about 95%, or at least about 98% identity to SEQ ID NO: 32 or 42. In some embodiments, the anti-human λ light chain antibodies and/or human λ light chain binding antibody fragments bind an epitope comprising SEQ ID NO: 51.

Also provided are provided and described herein are anti-human λ light chain antibodies and/or human λ light chain binding antibody fragments comprising:
(a) a heavy chain complementarity determining region 1 (H-CDR1) comprising an amino acid sequence NFGMH;
(b) a heavy chain complementarity determining region 2 (H-CDR2) comprising an amino acid sequence WMNTYTGESTYAD(D or E)FKG;
(c) a heavy chain complementarity determining region 3 (H-CDR3) comprising an amino acid sequence ISYDYDGYTMD(F or Y);
(d) a light chain complementarity determining region 1 (L-CDR1) comprising an amino acid sequence RS(G or S)QSLVH(R or S)NGNTY(F or L)H;
(e) a light chain complementarity determining region 2 (L-CDR2) comprising an amino acid sequence K(V or L)SI(R or N)FS; and/or
(f) a light chain complementarity determining region 3 (L-CDR3) comprising an amino acid sequence SQSTHVPWT,
   wherein the anti-human λ light chain antibodies and/or human λ light chain binding antibody fragments bind an epitope comprising SEQ ID NO: 51. In some embodiments, the anti-human λ light chain antibodies and/or human λ light chain binding antibody fragments comprise: a heavy chain variable region comprising an amino acid sequence having at least about 70%, at least about 80%, at least about 85%, at least about 90%, at least about 95%, or at least about 98% identity to SEQ ID NO: 31 or 41; and/or a light chain variable region comprises an amino acid sequence having at least about 70%, at least about 80%, at least about 85%, at least about 90%, at least about 95%, or at least about 98% identity to SEQ ID NO: 32 or 42. In some embodiments, the anti-human λ light chain antibodies and/or human λ light chain binding antibody fragments bind an epitope comprising SEQ ID NO: 51.

Provided and described herein are anti-human λ light chain antibodies and/or human λ light chain binding antibody fragments comprising:
(a) a heavy chain complementarity determining region 1 (H-CDR1);
(b) a heavy chain complementarity determining region 2 (H-CDR2);
(c) a heavy chain complementarity determining region 3 (H-CDR3);
(d) a light chain complementarity determining region 1 (L-CDR1);
(e) a light chain complementarity determining region 2 (L-CDR2); and/or
(f) a light chain complementarity determining region 3 (L-CDR3);
   wherein the H-CDR1, the H-CDR2, and the H-CDR3 are derived from SEQ ID NO: 1, and the L-CDR1, the L-CDR2, and the L-CDR3 derived from SEQ ID NO: 2; and wherein the H-CDR1, the H-CDR2, the H-CDR3, L-CDR1, the L-CDR2, and the L-CDR3 are defined using Chothia, Kabat, IMGT, Contact, or AbM methodologies. In some embodiments, the H-CDR1, the H-CDR2, the H-CDR3, L-CDR1, the L-CDR2, and the L-CDR3 are defined using Chothia. In some embodiments, the H-CDR1, the H-CDR2, the H-CDR3, L-CDR1, the L-CDR2, and the L-CDR3 are defined using Kabat. In some embodiments, the H-CDR1, the H-CDR2, the H-CDR3, L-CDR1, the L-CDR2, and the L-CDR3 are defined using IMGT. In some embodiments, the H-CDR1, the H-CDR2, the H-CDR3, L-CDR1, the L-CDR2, and the L-CDR3 are defined using Contact. In some embodiments, the H-CDR1, the H-CDR2, the H-CDR3, L-CDR1, the L-CDR2, and the L-CDR3 are defined using AbM.

Provided and described herein are anti-human λ light chain antibodies and/or human λ light chain binding antibody fragments comprising:
(a) a heavy chain complementarity determining region 1 (H-CDR1);
(b) a heavy chain complementarity determining region 2 (H-CDR2);
(c) a heavy chain complementarity determining region 3 (H-CDR3);
(d) a light chain complementarity determining region 1 (L-CDR1);
(e) a light chain complementarity determining region 2 (L-CDR2); and/or
(f) a light chain complementarity determining region 3 (L-CDR3);
   wherein the H-CDR1, the H-CDR2, and the H-CDR3 are derived from SEQ ID NO: 11, and the L-CDR1, the L-CDR2, and the L-CDR3 derived from SEQ ID NO: 12; and wherein the H-CDR1, the H-CDR2, the H-CDR3, L-CDR1, the L-CDR2, and the L-CDR3 are defined using Chothia, Kabat, IMGT, Contact, or AbM methodologies. In some embodiments, the H-CDR1, the H-CDR2, the H-CDR3, L-CDR1, the L-CDR2, and the L-CDR3 are defined using Chothia. In some embodiments, the H-CDR1, the H-CDR2, the H-CDR3, L-CDR1, the L-CDR2, and the L-CDR3 are defined using Kabat. In some embodiments, the H-CDR1, the H-CDR2, the H-CDR3, L-CDR1, the L-CDR2, and the L-CDR3 are defined using IMGT. In some embodiments, the H-CDR1, the H-CDR2, the H-CDR3, L-CDR1, the L-CDR2, and the L-CDR3 are defined using Contact. In some embodiments, the H-CDR1, the H-CDR2, the H-CDR3, L-CDR1, the L-CDR2, and the L-CDR3 are defined using AbM.

Provided and described herein are anti-human λ light chain antibodies and/or human λ light chain binding antibody fragments comprising:
(a) a heavy chain complementarity determining region 1 (H-CDR1);
(b) a heavy chain complementarity determining region 2 (H-CDR2);
(c) a heavy chain complementarity determining region 3 (H-CDR3);
(d) a light chain complementarity determining region 1 (L-CDR1);
(e) a light chain complementarity determining region 2 (L-CDR2); and/or
(f) a light chain complementarity determining region 3 (L-CDR3);
   wherein the H-CDR1, the H-CDR2, and the H-CDR3 are derived from SEQ ID NO: 21, and the L-CDR1, the L-CDR2, and the L-CDR3 derived from SEQ ID NO: 22; and wherein the H-CDR1, the H-CDR2, the H-CDR3, L-CDR1, the L-CDR2, and the L-CDR3 are defined using Chothia, Kabat, IMGT, Contact, or AbM methodologies. In some embodiments, the H-CDR1, the H-CDR2, the H-CDR3, L-CDR1, the L-CDR2, and the L-CDR3 are defined using Chothia. In some embodiments, the H-CDR1, the H-CDR2, the H-CDR3, L-CDR1, the L-CDR2, and the L-CDR3 are defined using Kabat. In some embodiments, the H-CDR1, the H-CDR2, the H-CDR3, L-CDR1, the L-CDR2, and the L-CDR3 are defined using IMGT. In some embodiments, the H-CDR1, the H-CDR2, the H-CDR3, L-CDR1, the L-CDR2, and the L-CDR3 are defined using Contact. In some embodiments, the H-CDR1, the H-CDR2, the H-CDR3, L-CDR1, the L-CDR2, and the L-CDR3 are defined using AbM.

Provided and described herein are anti-human λ light chain antibodies and/or human λ light chain binding antibody fragments comprising:
(a) a heavy chain complementarity determining region 1 (H-CDR1);
(b) a heavy chain complementarity determining region 2 (H-CDR2);
(c) a heavy chain complementarity determining region 3 (H-CDR3);
(d) a light chain complementarity determining region 1 (L-CDR1);
(e) a light chain complementarity determining region 2 (L-CDR2); and/or
(f) a light chain complementarity determining region 3 (L-CDR3);
   wherein the H-CDR1, the H-CDR2, and the H-CDR3 are derived from SEQ ID NO: 31, and the L-CDR1, the L-CDR2, and the L-CDR3 derived from SEQ ID NO: 32; and wherein the H-CDR1, the H-CDR2, the H-CDR3, L-CDR1, the L-CDR2, and the L-CDR3 are defined using Chothia, Kabat, IMGT, Contact, or AbM methodologies. In some embodiments, the H-CDR1, the H-CDR2, the H-CDR3, L-CDR1, the L-CDR2, and the L-CDR3 are defined using Chothia. In some embodiments, the H-CDR1, the H-CDR2, the H-CDR3, L-CDR1, the L-CDR2, and the L-CDR3 are defined using Kabat. In some embodiments, the H-CDR1, the H-CDR2, the H-CDR3, L-CDR1, the L-CDR2, and the L-CDR3 are defined using IMGT. In some embodiments, the H-CDR1, the H-CDR2, the H-CDR3, L-CDR1, the L-CDR2, and the L-CDR3 are defined using Contact. In some embodiments, the H-CDR1, the H-CDR2, the H-CDR3, L-CDR1, the L-CDR2, and the L-CDR3 are defined using AbM.

Provided and described herein are anti-human λ light chain antibodies and/or human λ light chain binding antibody fragments comprising:
(a) a heavy chain complementarity determining region 1 (H-CDR1);
(b) a heavy chain complementarity determining region 2 (H-CDR2);
(c) a heavy chain complementarity determining region 3 (H-CDR3);
(d) a light chain complementarity determining region 1 (L-CDR1);
(e) a light chain complementarity determining region 2 (L-CDR2); and/or
(f) a light chain complementarity determining region 3 (L-CDR3);
   wherein the H-CDR1, the H-CDR2, and the H-CDR3 are derived from SEQ ID NO: 41, and the L-CDR1, the L-CDR2, and the L-CDR3 derived from SEQ ID NO: 42; and wherein the H-CDR1, the H-CDR2, the H-CDR3, L-CDR1, the L-CDR2, and the L-CDR3 are defined using Chothia, Kabat, IMGT, Contact, or AbM methodologies. In some embodiments, the H-CDR1, the H-CDR2, the H-CDR3, L-CDR1, the L-CDR2, and the L-CDR3 are defined using Chothia. In some embodiments, the H-CDR1, the H-CDR2, the H-CDR3, L-CDR1, the L-CDR2, and the L-CDR3 are defined using Kabat. In some embodiments, the H-CDR1, the H-CDR2, the H-CDR3, L-CDR1, the L-CDR2, and the L-CDR3 are defined using IMGT. In some embodiments, the H-CDR1, the H-CDR2, the H-CDR3, L-CDR1, the L-CDR2, and the L-CDR3 are defined using Contact. In some embodiments, the H-CDR1, the H-CDR2, the H-CDR3, L-CDR1, the L-CDR2, and the L-CDR3 are defined using AbM.

In some embodiments, the anti-human λ light chain antibodies and/or human λ light chain binding antibody fragments bind to an epitope comprising SEQ ID NO: 1. In certain embodiments, the anti-human λ light chain antibodies and/or human λ light chain binding antibody fragments bind to an epitope consisting of SEQ ID NO: 1. In certain embodiments, binding is measured by ELISA, surface plasmon resonance, bio-layer interferometry, or isothermal calorimetry.

In some embodiments, the anti-human λ light chain antibodies and/or human λ light chain binding antibody fragments further comprise a heavy chain framework region 1 (H-FR1), a heavy chain framework region 2 (H-FR2), a heavy chain framework region 3 (H-FR3), and a heavy chain framework region 4 (H-FR4); and a light chain framework region 1 (L-FR1), a light chain framework region 2 (L-FR2), a light chain framework region 3 (L-FR3), and a light chain framework region 4 (L-FR4). In some embodiments, the anti-human λ light chain antibodies and/or human λ light chain binding antibody fragments further comprise a heavy chain variable region and a light chain variable region. In some embodiments, the anti-human λ light chain antibodies and/or human λ light chain binding antibody fragments further comprise a heavy chain constant region and a light chain constant region.

### Anti-Human λ Light Chain Variable Regions

Provided and described herein are anti-human λ light chain antibodies and/or human λ light chain binding antibody fragments comprising:
a heavy chain variable region comprising an amino acid sequence having at least about 70% identity to SEQ ID NO: 1; and/or
a light chain variable region comprising an amino acid sequence having at least about 70% identity to SEQ ID NO: 2;
   wherein the anti-human λ light chain antibodies and/or human λ light chain binding antibody fragments bind an epitope comprising SEQ ID NO: 51 (e.g., as measured by an ELISA). In some embodiments, the heavy chain variable region comprises an amino acid sequence having at least about 80% identity to SEQ ID NO: 1, and the light chain variable region comprises an amino acid sequence having at least about 80% identity to SEQ ID NO: 2. In some embodiments, the heavy chain variable region comprises an amino acid sequence having at least about 85% identity to SEQ ID NO: 1, and the light chain variable region comprises an amino acid sequence having at least about 85% identity to SEQ ID NO: 2. In some embodiments, the heavy chain variable region comprises an amino acid sequence having at least about 90% identity to SEQ ID NO: 1, and the light chain variable region comprises an amino acid sequence having at least about 90% identity to SEQ ID NO: 2. In some embodiments, the heavy chain variable region comprises an amino acid sequence having at least about 95% identity to SEQ ID NO: 1, and the light chain variable region comprises an amino acid sequence having at least about 95% identity to SEQ ID NO: 2. In some embodiments, the heavy chain variable region comprises an amino acid sequence having at least about 97% identity to SEQ ID NO: 1, and the light chain variable region comprises an amino acid sequence having at least about 97% identity to SEQ ID NO: 2. In some embodiments, the heavy chain variable region comprises an amino acid sequence having at least about 98% identity to SEQ ID NO: 1, and the light chain variable region comprises an amino acid sequence having at least about 98% identity to SEQ ID NO: 2. In some embodiments, the heavy chain variable region comprises an amino acid sequence having at least about 99% identity to SEQ ID NO: 1, and the light chain variable region comprises an amino acid sequence having at least about 99% identity to SEQ ID NO: 2.

Also provided and described herein are anti-human λ light chain antibodies and/or human λ light chain binding antibody fragments comprising:
a heavy chain variable region comprising an amino acid sequence having at least about 70% identity to SEQ ID NO: 11; and/or
a light chain variable region comprising an amino acid sequence having at least about 70% identity to SEQ ID NO: 12;
   wherein the anti-human λ light chain antibodies and/or human λ light chain binding antibody fragments bind an epitope comprising SEQ ID NO: 51 (e.g., as measured by an ELISA). In some embodiments, the heavy chain variable region comprises an amino acid sequence having at least about 80% identity to SEQ ID NO: 11, and the light chain variable region comprises an amino acid sequence having at least about 80% identity to SEQ ID NO: 12. In some embodiments, the heavy chain variable region comprises an amino acid sequence having at least about 85% identity to SEQ ID NO: 11, and the light chain variable region comprises an amino acid sequence having at least about 85% identity to SEQ ID NO: 12. In some embodiments, the heavy chain variable region comprises an amino acid sequence having at least about 90% identity to SEQ ID NO: 11, and the light chain variable region comprises an amino acid sequence having at least about 90% identity to SEQ ID NO: 12. In some embodiments, the heavy chain variable region comprises an amino acid sequence having at least about 95% identity to SEQ ID NO: 11, and the light chain variable region comprises an amino acid sequence having at least about 95% identity to SEQ ID NO: 12. In some embodiments, the heavy chain variable region comprises an amino acid sequence having at least about 97% identity to SEQ ID NO: 11, and the light chain variable region comprises an amino acid sequence having at least about 97% identity to SEQ ID NO: 12. In some embodiments, the heavy chain variable region comprises an amino acid sequence having at least about 98% identity to SEQ ID NO: 11, and the light chain variable region comprises an amino acid sequence having at least about 98% identity to SEQ ID NO: 12. In some embodiments, the heavy chain variable region comprises an amino acid sequence having at least about 99% identity to SEQ ID NO: 11, and the light chain variable region comprises an amino acid sequence having at least about 99% identity to SEQ ID NO: 12.

Provided and described herein are anti-human λ light chain antibodies and/or human λ light chain binding antibody fragments comprising:
a heavy chain variable region comprising an amino acid sequence having at least about 70% identity to SEQ ID NO: 21; and/or
a light chain variable region comprising an amino acid sequence having at least about 70% identity to SEQ ID NO: 22;
   wherein the anti-human λ light chain antibodies and/or human λ light chain binding antibody fragments bind an epitope comprising SEQ ID NO: 51 (e.g., as measured by an ELISA). In some embodiments, the heavy chain variable region comprises an amino acid sequence having at least about 80% identity to SEQ ID NO: 21, and the light chain variable region comprises an amino acid sequence having at least about 80% identity to SEQ ID NO: 22. In some embodiments, the heavy chain variable region comprises an amino acid sequence having at least about 85% identity to SEQ ID NO: 21, and the light chain variable region comprises an amino acid sequence having at least about 85% identity to SEQ ID NO: 22. In some embodiments, the heavy chain variable region comprises an amino acid sequence having at least about 90% identity to SEQ ID NO: 1, and the light chain variable region comprises an amino acid sequence having at least about 90% identity to SEQ ID NO: 22. In some embodiments, the heavy chain variable region comprises an amino acid sequence having at least about 95% identity to SEQ ID NO: 21, and the light chain variable region comprises an amino acid sequence having at least about 95% identity to SEQ ID NO: 22. In some embodiments, the heavy chain variable region comprises an amino acid sequence having at least about 97% identity to SEQ ID NO: 21, and the light chain variable region comprises an amino acid sequence having at least about 97% identity to SEQ ID NO: 22. In some embodiments, the heavy chain variable region comprises an amino acid sequence having at least about 98% identity to SEQ ID NO: 21, and the light chain variable region comprises an amino acid sequence having at least about 98% identity to SEQ ID NO: 22. In some embodiments, the heavy chain variable region comprises an amino acid sequence having at least about 99% identity to SEQ ID NO: 21, and the light chain variable region comprises an amino acid sequence having at least about 99% identity to SEQ ID NO: 22.

Provided and described herein are anti-human λ light chain antibodies and/or human λ light chain binding antibody fragments comprising:
a heavy chain variable region comprising an amino acid sequence having at least about 70% identity to SEQ ID NO: 31; and/or
a light chain variable region comprising an amino acid sequence having at least about 70% identity to SEQ ID NO: 32;
   wherein the anti-human λ light chain antibodies and/or human λ light chain binding antibody fragments bind an epitope comprising SEQ ID NO: 51 (e.g., as measured by an ELISA). In some embodiments, the heavy chain variable region comprises an amino acid sequence having at least about 80% identity to SEQ ID NO: 31, and the light chain variable region comprises an amino acid sequence having at least about 80% identity to SEQ ID NO: 32. In some embodiments, the heavy chain variable region comprises an amino acid sequence having at least about 85% identity to SEQ ID NO: 31, and the light chain variable region comprises an amino acid sequence having at least about 85% identity to SEQ ID NO: 32. In some embodiments, the heavy chain variable region comprises an amino acid sequence having at least about 90% identity to SEQ ID NO: 31, and the light chain variable region comprises an amino acid sequence having at least about 90% identity to SEQ ID NO: 32. In some embodiments, the heavy chain variable region comprises an amino acid sequence having at least about 95% identity to SEQ ID NO: 31, and the light chain variable region comprises an amino acid sequence having at least about 95% identity to SEQ ID NO: 32. In some embodiments, the heavy chain variable region comprises an amino acid sequence having at least about 97% identity to SEQ ID NO: 31, and the light chain variable region comprises an amino acid sequence having at least about 97% identity to SEQ ID NO: 32. In some embodiments, the heavy chain variable region comprises an amino acid sequence having at least about 98% identity to SEQ ID NO: 31, and the light chain variable region comprises an amino acid sequence having at least about 98% identity to SEQ ID NO: 32. In some embodiments, the heavy chain variable region comprises an amino acid sequence having at least about 99% identity to SEQ ID NO: 31, and the light chain variable region comprises an amino acid sequence having at least about 99% identity to SEQ ID NO: 32.

Provided and described herein are anti-human λ light chain antibodies and/or human λ light chain binding antibody fragments comprising:
a heavy chain variable region comprising an amino acid sequence having at least about 70% identity to SEQ ID NO: 41; and/or
a light chain variable region comprising an amino acid sequence having at least about 70% identity to SEQ ID NO: 42;
   wherein the anti-human λ light chain antibodies and/or human λ light chain binding antibody fragments bind an epitope comprising SEQ ID NO: 51 (e.g., as measured by an ELISA). In some embodiments, the heavy chain variable region comprises an amino acid sequence having at least about 80% identity to SEQ ID NO: 41, and the light chain variable region comprises an amino acid sequence having at least about 80% identity to SEQ ID NO: 42. In some embodiments, the heavy chain variable region comprises an amino acid sequence having at least about 85% identity to SEQ ID NO: 41, and the light chain variable region comprises an amino acid sequence having at least about 85% identity to SEQ ID NO: 42. In some embodiments, the heavy chain variable region comprises an amino acid sequence having at least about 90% identity to SEQ ID NO: 41, and the light chain variable region comprises an amino acid sequence having at least about 90% identity to SEQ ID NO: 42. In some embodiments, the heavy chain variable region comprises an amino acid sequence having at least about 95% identity to SEQ ID NO: 41, and the light chain variable region comprises an amino acid sequence having at least about 95% identity to SEQ ID NO: 42. In some embodiments, the heavy chain variable region comprises an amino acid sequence having at least about 97% identity to SEQ ID NO: 41, and the light chain variable region comprises an amino acid sequence having at least about 97% identity to SEQ ID NO: 42. In some embodiments, the heavy chain variable region comprises an amino acid sequence having at least about 98% identity to SEQ ID NO: 41, and the light chain variable region comprises an amino acid sequence having at least about 98% identity to SEQ ID NO: 42. In some embodiments, the heavy chain variable region comprises an amino acid sequence having at least about 99% identity to SEQ ID NO: 41, and the light chain variable region comprises an amino acid sequence having at least about 99% identity to SEQ ID NO: 42.

### Anti-Human λ Light Chain Constant Regions

In some embodiments, an anti-human λ light chain antibody encompasses intact or full-length antibodies, including antibodies of any class or sub-class, including IgG and sub-classes thereof, IgM, IgE, IgA, and IgD. In certain embodiments, the anti-human λ light chain antibodies comprise a human IgG constant region. In certain embodiments, the anti-human λ light chain antibodies comprise a human IgG1 constant region. In certain embodiments, the anti-human λ light chain antibodies comprise a human IgG2 constant region. In certain embodiments, the anti-human λ light chain antibodies comprise a human IgG3 constant region. In certain embodiments, the anti-human λ light chain antibodies comprise a human IgG4 constant region. In some embodiments, the anti-human λ light chain antibodies comprise a heavy chain sequence comprising at least 85% sequence identity to SEQ ID NO: 52. In some embodiments, the anti-human λ light chain antibodies comprise a heavy chain sequence comprising at least 90% sequence identity to SEQ ID NO: 52. In some embodiments, the anti-human λ light chain antibodies comprise a heavy chain sequence comprising at least 95% sequence identity to SEQ ID NO: 52. In some embodiments, the anti-human λ light chain antibodies comprise a heavy chain sequence comprising at least 98% sequence identity to SEQ ID NO: 52. In some embodiments, the anti-human λ light chain antibodies comprise a heavy chain sequence comprising at least 99% sequence identity to SEQ ID NO: 52. In some embodiments, the anti-human λ light chain antibodies comprise a heavy chain sequence comprising SEQ ID NO: 52.

Described herein in certain embodiments are antibodies that bind misfolded human λ light chains. In certain embodiments, the antibodies bind aggregated human λ light chains. In certain embodiments, the antibodies bind amyloid human λ light chains. In certain embodiments, the antibodies bind aggregated human λ light chains with a K_{D} of less than about 100 nanomolar. In certain embodiments, the antibodies bind aggregated human λ light chains with a K_{D} of less than about 90 nanomolar. In certain embodiments, the antibodies bind aggregated human λ light chains with a K_{D} of less than about 80 nanomolar. In certain embodiments, the antibodies bind aggregated human λ light chains with a K_{D} of greater than about 1, 10, 20, 30, 40, or 50 nanomolar. In certain embodiments, the antibodies bind amyloid human λ light chains with a K_{D} of less than about 100 nanomolar. In certain embodiments, the antibodies bind amyloid human λ light chains with a K_{D} of less than about 90 nanomolar. In certain embodiments, the antibodies bind amyloid human λ light chains with a K_{D} of less than about 80 nanomolar. In certain embodiments, the antibodies bind amyloid human λ light chains with a K_{D} of less than about 70 nanomolar. In certain embodiments, the antibodies bind amyloid human λ light chains with a K_{D} of greater than about 1, 10, 20, 30, 40, or 50 nanomolar.

### Compositions

Provided herein are compositions (e.g., pharmaceutical compositions) comprising the anti-human λ light chain antibodies and/or human λ light chain binding antibody fragments described herein. A composition or pharmaceutical formulation or pharmaceutical composition generally encompasses and/or refers to a preparation which is in such form as to permit the biological activity of an active ingredient contained therein to be effective, and which contains no additional components which are unacceptably toxic to a subject to which the formulation would be administered. Pharmaceutical formulations are generally sterile (e.g., aseptic or free from all living microorganisms and their spores).

Compositions comprising the anti-human λ light chain antibodies and/or human λ light chain binding antibody fragments described herein can include in a pharmaceutical composition comprising one or more pharmaceutically acceptable excipients, carriers, and diluents. In certain embodiments, the antibodies of the current disclosure are administered suspended in a sterile and/or isotonic solution. In certain embodiments, the solution comprises about 0.9% NaCl. In certain embodiments, the solution comprises about 5.0% dextrose. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, L-Histidine/L-histidine monohydrochloride monohydrate, trehalose, and dextran 40; amino acids, for example, glycine or arginine; antioxidants, for example, ascorbic acid, methionine; or chelating agents, for example, EDTA or EGTA.

Formulations for administration of antibodies can comprise one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine or arginine; antioxidants, for example, ascorbic acid, methionine; or chelating agents, for example, EDTA or EGTA. Additionally, a compound or molecule that relieves pain at the injection site can be included, such as hyaluronidase, for example at a concentration of from about 2,000 U/ml to about 12,000 U/ml.

In certain embodiments, the anti-human λ light chain antibodies or human λ light chain binding antibody fragments of described herein are shipped/stored lyophilized and reconstituted before administration. In certain embodiments, lyophilized antibody formulations comprise a bulking agent such as, mannitol, sorbitol, sucrose, trehalose, dextran 40, or combinations thereof. The lyophilized formulation can be contained in a vial comprised of glass or other suitable non-reactive material. The antibodies when formulated, whether reconstituted or not, can be buffered at a certain pH, generally less than 7.0. In certain embodiments, the pH can be between 4.5 and 6.5, 4.5 and 6.0, 4.5 and 5.5, 4.5 and 5.0, or 5.0 and 6.0.

In some embodiments, the anti-human λ light chain antibodies and/or human λ light chain binding antibody fragments are configured for intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural, or intrasternal injection. In some embodiments, the compositions comprising the anti-human λ light chain antibodies and/or human λ light chain binding antibody fragments is configured for intravenous injection.

Also described herein are kits comprising one or more of the antibodies described herein in a suitable container and one or more additional components selected from: instructions for use; a diluent, an excipient, a carrier, and a device for administration.

In certain embodiments, described herein is a method of preparing a treatment comprising admixing one or more pharmaceutically acceptable excipients, carriers, or diluents and the anti-human λ light chain antibodies and/or human λ light chain binding antibody fragments. In certain embodiments, described herein is a method of preparing a cancer treatment for storage or shipping comprising lyophilizing one or more antibodies of the current disclosure.

### Host Cells and Nucleic Acids

Host cells, host cell lines, and/or host cell cultures are used interchangeably, and generally include and/or refer to cells into which exogenous nucleic acid has been introduced, including the progeny of such cells. Host cells include transformants and transformed cells, which include the primary transformed cell and progeny derived therefrom without regard to the number of passages.

An isolated nucleic acid generally encompasses and/or refers to a nucleic acid molecule that has been separated from a component of its natural environment. An isolated nucleic acid encompasses a nucleic acid molecule contained in cells that ordinarily contain the nucleic acid molecule, but the nucleic acid molecule is present extrachromosomally or at a chromosomal location that is different from its natural chromosomal location. An Isolated nucleic acid encoding an antibody encompasses and/or refers to one or more nucleic acid molecules encoding antibody heavy and light chains (or fragments thereof), including such nucleic acid molecule(s) in a single vector or separate vectors, and such nucleic acid molecule(s) present at one or more locations in a host cell. In some embodiments, provided herein is a nucleic acid sequence encoding the anti-human λ light chain antibodies and/or human λ light chain binding antibody fragments described herein. In some embodiments, also provided is a host cell comprising the nucleic acid sequence encoding the anti-human λ light chain antibodies and/or human λ light chain binding antibody fragments. In some embodiments, the nucleic acid sequence encoding the anti-human λ light chain antibodies and/or human λ light chain binding antibody fragments comprises at least 60% sequence identity to any one of SEQ ID NOs: 9-10, 19-20, 29-30, 39-40, or 49-50. In some embodiments, the nucleic acid sequence encoding the anti-human λ light chain antibodies and/or human λ light chain binding antibody fragments comprises at least 70% sequence identity to any one of SEQ ID NOs: 9-10, 19-20, 29-30, 39-40, or 49-50. In some embodiments, the nucleic acid sequence encoding the anti-human λ light chain antibodies and/or human λ light chain binding antibody fragments comprises at least 80% sequence identity to any one of SEQ ID NOs: 9-10, 19-20, 29-30, 39-40, or 49-50. In some embodiments, the nucleic acid sequence encoding the anti-human λ light chain antibodies and/or human λ light chain binding antibody fragments comprises at least 90% sequence identity to any one of SEQ ID NOs: 9-10, 19-20, 29-30, 39-40, or 49-50. In some embodiments, the nucleic acid sequence encoding the anti-human λ light chain antibodies and/or human λ light chain binding antibody fragments comprises at least 95% sequence identity to any one of SEQ ID NOs: 9-10, 19-20, 29-30, 39-40, or 49-50. In some embodiments, the nucleic acid sequence encoding the anti-human λ light chain antibodies and/or human λ light chain binding antibody fragments comprises the sequence of any one of SEQ ID NOs: 9-10, 19-20, 29-30, 39-40, or 49-50.

### Methods

The anti-human λ light chain antibodies and/or human λ light chain binding antibody fragments described herein can be used to identify, detect, and treat misfolded λ light chain (e.g., λ light chain aggregates or amyloid deposits), and diseases or disorders associated therewith.

### Methods of Detecting Light Chains

Provided and described herein are method of diagnosing amyloidosis in a sample, the method comprising contacting a biological sample obtained from a subject with the anti-human λ light chain antibodies or human λ light chain binding antibody fragments described herein. Also provided herein are methods of detecting free light chain polypeptides in a sample, the method comprising contacting the biological sample with the anti-human λ light chain antibodies or human λ light chain binding antibody fragments described herein. Further provided are methods of detecting amyloid deposits in a sample, the method comprising contacting the biological sample with the anti-human λ light chain antibodies or human λ light chain binding antibody fragments described herein. In some embodiments, the sample is from blood, serum, plasma, solid tissue, and any combination thereof. In some embodiments, the sample is from a subject.

As described, certain embodiments of the present disclosure are directed to methods of measuring light chain immunoglobulins. The anti-human λ light chain antibodies or human λ light chain binding antibody fragments disclosed herein can be used to measure any immunoglobulin light chain or aggregate thereof. In some embodiments, the anti-human λ light chain antibodies or human λ light chain binding antibody fragments are used to measure an immunoglobulin light chain or an aggregate thereof in vivo. In some embodiments, the anti-human λ light chain antibodies or human λ light chain binding antibody fragments are used to measure an immunoglobulin light chain or an aggregate thereof in a biological sample collected from a subject. In some embodiments, the biological sample is a plasma sample, a blood sample, a tissue sample (e.g., a tissue biopsy), a urine sample, or any combination thereof.

In some embodiments, the method comprises measuring light chain fibrils. As such, the antibodies described herein can be used to identify a subject that has a disease or condition characterized by the formation of immunoglobulin light chain deposits, e.g., amyloid fibril deposits. In some embodiments, the anti-human λ light chain antibodies or human λ light chain binding antibody fragments are used to identify (e.g., diagnose) a subject as having an amyloidosis. In some embodiments, the anti-human λ light chain antibodies or human λ light chain binding antibody fragments are used to identify a subject as having a systemic amyloidosis. In some embodiments, the anti-human λ light chain antibodies or human λ light chain binding antibody fragments are used to identify a subject as having an AL amyloidosis. In some embodiments, the anti-human λ light chain antibodies or human λ light chain binding antibody fragments are used to identify a subject as having a plasma cell dyscrasia. In some embodiments, the anti-human λ light chain antibodies or human λ light chain binding antibody fragments are used to identify a subject as having a monoclonal gammopathy of unknown significance (MGUS). In some embodiments, the anti-human λ light chain antibodies or human λ light chain binding antibody fragments are used to identify a subject as having multiple myeloma (MM). In some embodiments, the anti-human λ light chain antibodies or human λ light chain binding antibody fragments are used to identify a subject as having plasma cell leukemia (PCL).

In some embodiments, the anti-human λ light chain antibody or human λ light chain binding antibody fragment comprises one or more labels, covalently or non-covalently antibody or antigen binding fragment. Any label known in the art can be used to increase the ability of anti-human λ light chain antibody or human λ light chain binding antibody fragment to be detected and/or visualized. In some embodiments, the label comprises a chemical label. In some embodiments, the label comprises an enzymatic label. In some embodiments, label comprises a substrate, which is detectable in the presence of a particular enzyme. In some embodiments, the label is a fluorescent label. In some embodiments, the label is a radioisotope label.

Certain embodiments of the present disclosure are directed to methods of measuring light chain immunoglobulins in vivo using an anti-human λ light chain antibody or human λ light chain binding antibody fragment disclosed herein. In some embodiments, the anti-human λ light chain antibody or human λ light chain binding antibody fragment comprises a detectable probe. In some embodiments, the anti-human λ light chain antibody or human λ light chain binding antibody fragment is a contrast-conjugated antibody. In some embodiments, the method is a diagnostic medical imaging method, e.g., a PET/CT, SPECT, MRI, or any combination thereof.

Methods of Treating and/or Reducing Misfolded a Light Chains

Provided and described herein are methods of promoting the clearance of a λ light chain aggregate by monocytic cells, the method comprising contacting the λ light chain aggregate with the anti-human λ light chain antibodies or human λ light chain binding antibody fragments described herein. Also provided and described are methods of increasing the clearance of a λ light chain aggregates, the method comprising contacting the λ light chain aggregate with the anti-human λ light chain antibodies or human λ light chain binding antibody fragments described herein. Further provided and described are methods of reducing λ light chain aggregates in a subject, the method comprising administering the anti-human λ light chain antibodies or human λ light chain binding antibody fragments described herein to the subject, thereby reducing the number of λ light chain aggregates in the subject. Provided and described are methods of treating a disease or condition characterized by λ light chain aggregates, the method comprising administering the anti-human λ light chain antibodies or human λ light chain binding antibody fragments described herein to the subject, thereby reducing the number of λ light chain aggregates in the subject. Provided and described are methods of treating AL amyloidosis (e.g., in a subject having λ light chain aggregates), the method comprising administering the anti-human λ light chain antibodies or human λ light chain binding antibody fragments described herein to the subject, thereby reducing the number of λ light chain aggregates in the subject.

In some embodiments, the subject has a plasma cell disorder. In some embodiments, the plasma cell disorder comprises plasma cell dyscrasia. In some embodiments, the plasma cell dyscrasia is selected from a monoclonal gammopathy of unknown significance (MGUS), multiple myeloma (MM), plasma cell leukemia (PCL), and any combination thereof. In certain embodiments, the subject is a human

Certain uses of the anti-human λ light chain antibodies or human λ light chain binding antibody fragments described herein are directed to methods of treating a disease or condition in a subject in need thereof, comprising administering an anti-human λ light chain antibody or human λ light chain binding antibody fragment described herein. In some embodiments, the disease or condition comprises an amyloidosis. In some embodiments, the disease or condition comprises a systemic amyloidosis. In some embodiments, the disease or condition comprises an AL amyloidosis. In some embodiments, the disease or condition comprises a plasma cell dyscrasia. In some embodiments, the disease or condition comprises a monoclonal gammopathy of unknown significance (MGUS). In some embodiments, the disease or condition comprises multiple myeloma (MM). In some embodiments, the disease or condition comprises plasma cell leukemia (PCL).

Certain uses of the anti-human λ light chain antibodies or human λ light chain binding antibody fragments described herein are directed to methods of reducing and/or clearing an immunoglobulin light chain aggregate, e.g., an amyloid fibril, in a subject in need thereof, comprising administering an anti-human λ light chain antibody or human λ light chain binding antibody fragment described herein. In some embodiments, the subject has one or more immunoglobulin light chain aggregate, e.g., amyloid fibril, in the heart. In some embodiments, the subject has one or more immunoglobulin light chain aggregate, e.g., amyloid fibril, in a kidney. In some embodiments, the subject has one or more immunoglobulin light chain aggregate, e.g., amyloid fibril, in a tissue of the nervous system. In some embodiments, the subject has one or more immunoglobulin light chain aggregate, e.g., amyloid fibril, in the brain.

Certain uses of the anti-human λ light chain antibodies or human λ light chain binding antibody fragments described herein are directed to methods of treating a nephrotic syndrome in a subject in need thereof, comprising administering to the subject an anti-human λ light chain antibody or human λ light chain binding antibody fragment described herein, wherein the nephrotic syndrome comprises one or more immunoglobulin light chain deposit, e.g., one or more amyloid fibril deposit. Certain uses of the anti-human λ light chain antibodies or human λ light chain binding antibody fragments described herein are directed to methods of treating a hepatomegaly in a subject in need thereof, comprising administering to the subject an anti-human λ light chain antibody or human λ light chain binding antibody fragment described herein, wherein the hepatomegaly comprises one or more immunoglobulin light chain deposit, e.g., one or more amyloid fibril deposit. Certain uses of the anti-human λ light chain antibodies or human λ light chain binding antibody fragments described herein are directed to methods of treating a peripheral neuropathy in a subject in need thereof, comprising administering to the subject an anti-human λ light chain antibody or human λ light chain binding antibody fragment described herein, wherein the peripheral neuropathy comprises one or more immunoglobulin light chain deposit, e.g., one or more amyloid fibril deposit. Certain uses of the anti-human λ light chain antibodies or human λ light chain binding antibody fragments described herein are directed to methods of treating a restrictive cardiomyopathy in a subject in need thereof, comprising administering to the subject an anti-human λ light chain antibody or human λ light chain binding antibody fragment described herein, wherein the restrictive cardiomyopathy comprises one or more immunoglobulin light chain deposit, e.g., one or more amyloid fibril deposit.

In some embodiments, administering the anti-human λ light chain antibody or human λ light chain binding antibody fragment to the subject results in the reduction and/or clearance of one or more immunoglobulin light chain aggregate, e.g., amyloid fibril, in the subject. In some embodiments, the size of an immunoglobulin light chain aggregate, e.g., amyloid fibril, in the subject is reduced following administration of the anti-human λ light chain antibody or human λ light chain binding antibody fragment.

In certain embodiments, the antibodies can be administered to a subject in need thereof by any route suitable for the administration of antibody-containing pharmaceutical compositions, such as, for example, subcutaneous, intraperitoneal, intravenous, intramuscular, etc. In certain embodiments, the antibodies are administered intravenously. In certain embodiments, the antibodies are administered subcutaneously. In certain embodiments, the antibodies are administered on a suitable dosage schedule, for example, weekly, twice weekly, monthly, twice monthly, once every two weeks, once every three weeks, or once a month etc. In certain embodiments, the antibodies are administered once every three weeks. The antibodies can be administered in any therapeutically effective amount. In certain embodiments, therapeutically acceptable amount is between about 0.1 mg/kg and about 50 mg/kg. In certain embodiments, therapeutically acceptable amount is between about 1 mg/kg and about 40 mg/kg. In certain embodiments, therapeutically acceptable amount is between about 1 mg/kg and about 20 mg/kg. In certain embodiments, therapeutically acceptable amount is between about 1 mg/kg and about 10 mg/kg. In certain embodiments, therapeutically acceptable amount is between about 5 mg/kg and about 30 mg/kg. In certain embodiments, therapeutically acceptable amount is between about 5 mg/kg and about 20 mg/kg. Therapeutically effective amounts include amounts sufficient to ameliorate one or more symptoms associated with the disease or affliction to be treated.

### Pharmaceutically Acceptable Excipients, Carriers, and Diluents

In certain embodiments the anti-lambda light chain antibodies of the current disclosure are included in a pharmaceutical composition comprising one or more pharmaceutically acceptable excipients, carriers, and diluents. Pharmaceutically acceptable excipients, carriers and diluents can be included to increase shelf-life, stability, or the administrability of the antibody. Such compounds include salts, pH buffers, detergents, anti-coagulants, and preservatives. In certain embodiments, the antibodies of the current disclosure are administered suspended in a sterile solution. In certain embodiments, the solution comprises about 0.9% NaCl. In certain embodiments, the solution comprises about 5.0% dextrose. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine or arginine; antioxidants, for example, ascorbic acid, methionine; or chelating agents, for example, EDTA or EGTA.

In certain embodiments, the antibodies of the current disclosure can be shipped/stored lyophilized and reconstituted before administration. In certain embodiments, lyophilized antibody formulations comprise a bulking agent such as, mannitol, sorbitol, sucrose, trehalose, dextran 40, or combinations thereof. The lyophilized formulation can be contained in a vial comprised of glass or other suitable non-reactive material. The antibodies when formulated, whether reconstituted or not, can be buffered at a certain pH, generally less than 7.0. In certain embodiments, the pH can be between 4.5 and 7.0, 4.5 and 6.5, 4.5 and 6.0, 4.5 and 5.5, 4.5 and 5.0, or 5.0 and 6.0.

Also described herein are kits comprising one or more of the antibodies described herein in a suitable container and one or more additional components selected from: instructions for use; a diluent, an excipient, a carrier, and a device for administration.

In certain embodiments, described herein is a method of preparing a lambda light chain amyloidosis treatment comprising admixing one or more pharmaceutically acceptable excipients, carriers, or diluents and an antibody of the current disclosure. In certain embodiments, described herein is a method of preparing a cancer treatment for storage or shipping comprising lyophilizing one or more antibodies of the current disclosure.

### Definitions

An antibody is used in the broadest sense, and generally encompasses and/or refers to various antibody structures, including but not limited to monoclonal antibodies, polyclonal antibodies, multispecific antibodies (e.g., bispecific antibodies), and antibody fragments so long as they exhibit the desired antigen-binding activity. In some embodiments, an antibody or antibodies include intact antibodies and functional (antigen-binding) antibody fragments, including fragment antigen binding (Fab) fragments, F(ab′)₂ fragments, Fab′ fragments, Fv fragments, recombinant IgG (rIgG) fragments, single chain antibody fragments, including single chain variable fragments (sFv or scFv), and single domain antibodies (e.g., sdAb, sdFv, nanobody) fragments. In some embodiments, an antibody or antibodies include genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. In some embodiments, an antibody or antibodies encompass functional antibody fragments. In some embodiments, an antibody or antibodies encompasses intact or full-length antibodies, including antibodies of any class or sub-class, including IgG and sub-classes thereof, IgM, IgE, IgA, and IgD. The antibody can comprise a human IgG1 constant region. The antibody can comprise a human IgG4 constant region. In some embodiments, an antibody or antibodies include, but is not limited to, full-length and native antibodies, as well as fragments and portion thereof retaining the binding specificities thereof, such as any specific binding portion thereof including those having any number of, immunoglobulin classes and/or isotypes (e.g., IgG1, IgG2, IgG3, IgG4, IgM, IgA, IgD, IgE and IgM); and biologically relevant (antigen-binding) fragments or specific binding portions thereof, including but not limited to Fab, F(ab′)₂, Fv, and scFv (single chain or related entity). A monoclonal antibody is generally one within a composition of substantially homogeneous antibodies; thus, any individual antibodies comprised within the monoclonal antibody composition are identical except for possible naturally occurring mutations that can be present within a composition in minor amounts. A monoclonal antibody can comprise a human IgG1 constant region or a human IgG4 constant region.

A native antibody generally encompasses and/or refers to naturally occurring immunoglobulin molecules with varying structures. For example, native IgG antibodies are heterotetrameric glycoproteins of about 150,000 Daltons, composed of two identical light chains and two identical heavy chains that are disulfide bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody can be assigned to one of two types, called kappa (κ) and lambda (λ), based on the amino acid sequence of its constant domain.

A full-length antibody, intact antibody, and whole antibody are interchangeable, and generally include and/or refer to an antibody having a structure substantially similar to a native antibody structure or having heavy chains that contain an Fc region as defined herein.

A human consensus framework generally encompasses and/or refers to a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In some embodiments, for the VL, the subgroup is subgroup kappa I as in Kabat et al., supra. In some embodiments, for the VH, the subgroup is subgroup III as in Kabat et al., supra. A humanized antibody encompasses and refers to a chimeric antibody comprising amino acid residues from non-human HVRs and amino acid residues from human framework regions (FRs). In some embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally encompasses at least a portion of an antibody constant region derived from a human antibody. A “humanized form” of an antibody, e.g., a non-human antibody, refers to an antibody that has undergone humanization.

A complementarity determining region or CDR, which are synonymous with hypervariable region or HVR, generally include and/or refer to regions of an antibody which are hypervariable in sequence and/or form structurally defined loops (hypervariable loops) and/or contain the antigen-contacting residues (antigen contacts).

In some embodiments, complementarity determining regions or CDRs generally include and refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3).

A framework region or FR generally includes and/or refers to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). In some embodiments, the framework regions are defined by the non-CDR sequences of a variable region sequence. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Pluckthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg N R and Rees A R, “WAM: an improved algorithm for modelling antibodies on the WEB,” Protein Eng. 2000 December; 13(12):819-24 (“AbM” numbering scheme. In certain embodiments, the CDRs of the antibodies described herein can be defined by a method selected from Kabat, Chothia, IMGT, Aho, AbM, or combinations thereof. In some embodiments, the CDRs and FRs are defined by and/or according to a Kabat numbering scheme. In some embodiments, the CDRs and FRs are defined by and/or according to a Chothia numbering scheme. In some embodiments, the CDRs and FRs are defined by and/or according to a IMGT numbering scheme. In some embodiments, the CDRs and FRs are defined by and/or according to an EU numbering scheme.

The boundaries of a given CDR or FR, in certain instances, vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

Fc region generally encompasses and/or refers to a C-terminal region of an immunoglobulin heavy chain that contains at least a portion of the constant region. The term includes native sequence Fc regions and variant Fc regions. In some embodiments, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

A human antibody generally encompasses and/or refers to an antibody which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

An acceptor human framework region generally encompasses and/or refers to a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework can comprise the same amino acid sequence thereof, or it can contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

A variable region or variable domain generally encompasses and/or refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs (See e.g., Kindt et al. Kuby Immunology, 6th ed., W.H. Freeman and Co., page 91(2007)). A single V_{H} or V_{L} domain can be sufficient to confer antigen-binding specificity. Furthermore, antibodies that bind a particular antigen can be isolated using a V_{H} or V_{L} domain from an antibody that binds the antigen to screen a library of complementary V_{L} or V_{H} domains, respectively (See e.g., Portolano et al., J. Immunol. 150:880-887 (1993); Clarkson et al., Nature 352:624-628 (1991)).

Affinity generally encompasses and/or refers to the strength of the sum total of noncovalent interactions between a single binding site of a molecule (e.g., an antibody) and its binding partner (e.g., an antigen). Unless indicated otherwise, as used herein, binding affinity generally encompasses and refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary embodiments for measuring binding affinity are described throughout.

An affinity matured antibody generally encompasses and/or refers to an antibody with one or more alterations in one or more hypervariable regions (HVRs), compared to a parent antibody which does not possess such alterations, such alterations resulting in an improvement in the affinity of the antibody for antigen.

Binding and a determination of binding (e.g., to an epitope comprising SEQ ID NO: 51) can be readily determined by methods known within the art (e.g., ELISA, surface plasmon resonance, bio-layer interferometry, isothermal calorimetry, etc.). In some embodiments, binding is determined by ELISA. In some embodiments, binding comprising a K_{D} less than, e.g., 10\hat{ }5 M (10 uM) as measured by surface plasmon resonance, bio-layer interferometry, or isothermal calorimetry. In some embodiments, binding comprising a K_{D} less than, e.g., 10\hat{ }6 M (1 uM) surface plasmon resonance, bio-layer interferometry, or isothermal calorimetry. In some embodiments, binding comprising a K_{D} less than, e.g., 10\hat{ }7 M (100 nM) surface plasmon resonance, bio-layer interferometry, or isothermal calorimetry.

A chimeric antibody generally encompasses and/or refers to an antibody in which a portion of the heavy and/or light chain is derived from a particular source or species, while the remainder of the heavy and/or light chain is derived from a different source or species. 1 The “class” of an antibody refers to the type of constant domain or constant region possessed by its heavy chain. There are five major classes of antibodies: IgA, IgD, IgE, IgG, and IgM, and several of these can be further divided into subclasses (isotypes), e.g., IgG₁, IgG₂, IgG₃, IgG₄, IgA₁, and IgA₂. The heavy chain constant domains that correspond to the different classes of immunoglobulins are called a, d, e, g, and m, respectively.

A monoclonal antibody generally encompasses and/or refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present within a composition in minor amounts. In contrast to polyclonal antibody preparations, which typically include different antibodies directed against different determinants (epitopes), each monoclonal antibody of a monoclonal antibody preparation is directed against a single determinant on an antigen. In some embodiments, the modifier monoclonal indicates the character of the antibody as being obtained from a substantially homogeneous population of antibodies and is not to be construed as requiring production of the antibody by any particular method. For example, the monoclonal antibodies to be used in accordance with the present within a composition disclosure can be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

An isolated antibody generally encompasses and/or refers to an antibody that has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC).

In certain embodiments, the antibody comprises one or more naturally occurring amino acids. In certain embodiments, the antibody consists of naturally occurring amino acids. As used herein, naturally occurring amino acids include and/or refer to amino acids which are generally found in nature and are not manipulated by man. In certain instances, naturally occurring includes and/or further refers to the 20 conventional amino acids: alanine (A or Ala), cysteine (C or Cys), aspartic acid (D or Asp), glutamic acid (E or Glu), phenylalanine (F or Phe), glycine (G or Gly), histidine (H or His), isoleucine (I or Ile), lysine (K or Lys), leucine (L or Leu), methionine (M or Met), asparagine (N or Asn), proline (P or Pro), glutamine (Q or Gln), arginine (R or Arg), serine (S or Ser), threonine (T or Thr), valine (V or Val), tryptophan (W or Trp), and tyrosine (Y or Tyr).

In some embodiments, the antibody comprises a variant sequence of the antibody. In certain instances, amino acid substitutions can be made in the sequence of any of the antibodies described herein, without necessarily decreasing or ablating its activity (as measured by, e.g., the binding or functional assays described herein). Accordingly, in some embodiments, the variant sequence comprises one or more amino acid substitutions (e.g., within the variable region or within one or more CDRs). In some embodiments, the variant sequence comprises one or more substitutions in one or more CDRs. In certain embodiments, the variant sequence comprises one amino acid substitution. In certain embodiments, the variant sequence comprises two amino acid substitutions. In certain embodiments, the variant sequence comprises three amino acid substitutions. In certain instances, substitutions include conservative substitutions (e.g., substitutions with amino acids of comparable chemical characteristics). In certain instances, a non-polar amino acid can be substituted and replaced with another non-polar amino acid, wherein non-polar amino acids include alanine, leucine, isoleucine, valine, glycine, proline, phenylalanine, tryptophan and methionine. In certain instances, a neutrally charged polar amino acids can be substituted and replaced with another neutrally charged polar amino acid, wherein neutrally charged polar amino acids include serine, threonine, cysteine, tyrosine, asparagine, and glutamine. In certain instances, a positively charged amino acid can be substituted and replaced with another positively charged amino acid, wherein positively charged amino acids include arginine, lysine and histidine. In certain instances, a negatively charged amino acid can be substituted and replaced with another negatively charged amino acid, wherein negatively charged amino acids include aspartic acid and glutamic acid. Examples of amino acid substitutions also include substituting an L-amino acid for its corresponding D-amino acid, substituting cysteine for homocysteine or other non-natural amino acids.

In certain embodiments, the antibody comprises one or more non-natural amino acids. In certain embodiments, the antibody consists of non-natural amino acids. As used herein, non-natural amino acids and/or unnatural amino acids include and/or refer to amino acid structures that cannot be generated biosynthetically in any organism using unmodified or modified genes from any organism. For example, these include, but are not limited to, modified amino acids and/or amino acid analogues that are not one of the 20 naturally occurring amino acids (e.g., non-natural side chain variant sequence amino acids), D-amino acids, homo amino acids, beta-homo amino acids, N-methyl amino acids, alpha-methyl amino acids, or. By way of further example, non-natural amino acids also include 4-Benzoylphenylalanine (Bpa), Aminobenzoic Acid (Abz), Aminobutyric Acid (Abu), Aminohexanoic Acid (Ahx), Aminoisobutyric Acid (Aib), Citrulline (Cit), Diaminobutyric Acid (Dab), Diaminopropanoic Acid (Dap), Diaminopropionic Acid (Dap), Gamma-Carboxyglutamic Acid (Gla), Homoalanine (Hala), Homoarginine (Harg), Homoasparagine (Hasn), Homoaspartic Acid (Hasp), Homocysteine (Hcys), Homoglutamic Acid (Hglu), Homoglutamine (Hgln), Homoisoleucine (Hile), Homoleucine (Hleu), Homomethionine (Hmet), Homophenylalanine (Hphe), Homoserine (Hser), Homotyrosine (Htyr), Homovaline (Hval), Hydroxyproline (Hyp), Isonipecotic Acid (Inp), N aphthylalanine (Nal), Nipecotic Acid (Nip), Norleucine (Nle), Norvaline (Nva), Octahydroindole-2-carboxylic Acid (Oic), Penicillamine (Pen), Phenylglycine (Phg), Pyroglutamic Acid (Pyr), Sarcosine (Sar), tButylglycine (Tle), and Tetrahydro-isoquinoline-3-carboxylic Acid (Tic). Such non-natural amino acid residues can be introduced by substitution of naturally occurring amino acids, and/or by insertion of non-natural amino acids into the naturally occurring antibody sequence. A non-natural amino acid residue also can be incorporated such that a desired functionality is imparted to the apelin molecule, for example, the ability to link a functional moiety (e.g., PEG).

A stable formulation refers to and/or encompasses a formulation wherein the protein (e.g., antibody) therein essentially retains its physical stability and/or chemical stability and/or biological activity upon storage at an intended storage temperature, e.g., 2-8° C. In some embodiments, the formulation essentially retains its physical and chemical stability, as well as its biological activity upon storage. A storage period can generally be selected based on the intended shelf-life of the formulation. Furthermore, the formulation is generally stable following freezing (to, e.g., −20° C.) and thawing of the formulation, for example following 1 or more cycles of freezing and thawing. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993), for example. Stability can be measured at a selected temperature for a selected time period. Stability can be evaluated qualitatively and/or quantitatively in a variety of different ways, including evaluation of aggregate formation (for example using size exclusion chromatography, by measuring turbidity, and/or by visual inspection); by assessing charge heterogeneity using cation exchange chromatography or capillary zone electrophoresis; SDS-PAGE analysis to compare reduced and intact antibody; evaluating biological activity or antigen binding function of the antibody; and the methods described herein. Instability can involve any one or more of: aggregation, deamidation (e.g., Asn deamidation), oxidation (e.g. Met oxidation), isomerization (e.g. Asp isomeriation), clipping/hydrolysis/fragmentation (e.g. hinge region fragmentation), succinimide formation, unpaired cysteine(s), etc.

A pharmaceutically acceptable carrier generally encompasses and/or refers to an ingredient in a pharmaceutical formulation, other than an active ingredient, which is nontoxic to a subject. A pharmaceutically acceptable carrier encompasses, but is not limited to, a buffer, excipient, stabilizer, or preservative.

A polypeptide or protein are used interchangeably, and generally encompass and/or refer to a polymer of amino acid residues and are not limited to a minimum length. Polypeptides, including the provided antibodies and antibody chains and other peptides, e.g., linkers and binding peptides, can include amino acid residues including natural and/or non-natural amino acid residues. The terms also include post-expression modifications of the polypeptide, for example, glycosylation, sialylation, acetylation, phosphorylation, and the like. In some embodiments, the polypeptides can contain modifications with respect to a native or natural sequence, as long as the protein maintains the desired activity. These modifications can be deliberate, as through site-directed mutagenesis, or can be accidental, such as through mutations of hosts which produce the proteins or errors due to PCR amplification.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

As used herein the term individual, patient, or subject generally includes and/or refers to individuals diagnosed with, suspected of being afflicted with, or at-risk of developing at least one disease, condition, or status for which the described compositions and method are useful for treating. In certain embodiments, the individual is a mammal. In certain embodiments, the mammal is a mouse, rat, rabbit, dog, cat, horse, cow, sheep, pig, goat, llama, alpaca, or yak. In certain embodiments, the individual is a human.

The antibodies described herein can be encoded by a nucleic acid. A nucleic acid is a type of polynucleotide comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In the expression vectors regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a genomic region. In certain embodiments, the expression vector is a plasmid. In certain embodiments, the expression vector is a lentivirus, adenovirus, or adeno-associated virus. In certain embodiments, the expression vector is an adenovirus. In certain embodiments, the expression vector is an adeno-associated virus. In certain embodiments, the expression vector is a lentivirus.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). Percent homology of sequences can be determined using the most recent version of BLAST, as of the filing date of this application.

The nucleic acids encoding the antibodies described herein can be used to infect, transfect, transform, or otherwise render a suitable cell transgenic for the nucleic acid, thus enabling the production of antibodies for commercial or therapeutic uses. Standard cell lines and methods for the production of antibodies from a large-scale cell culture are known in the art. See e.g., Li et al., “Cell culture processes for monoclonal antibody production.” Mabs. 2010 September-October; 2(5): 466-477. In certain embodiments, the cell is a Eukaryotic cell. In certain embodiments, the Eukaryotic cell is a mammalian cell. In certain embodiments, the mammalian cell is a cell line useful for producing antibodies is a Chines Hamster Ovary cell (CHO) cell, an NS0 murine myeloma cell, or a PER.C6® cell. In certain embodiments, the nucleic acid encoding the antibody is integrated into a genomic locus of a cell useful for producing antibodies. In certain embodiments, described herein is a method of making an antibody comprising culturing a cell comprising a nucleic acid encoding an antibody under conditions in vitro sufficient to allow production and secretion of said antibody.

In certain embodiments, described herein, is a master cell bank comprising: (a) a mammalian cell line comprising a nucleic acid encoding an antibody described herein integrated at a genomic location; and (b) a cryoprotectant. In certain embodiments, the cryoprotectant comprises glycerol or DMSO. In certain embodiments, the master cell bank comprises: (a) a CHO cell line comprising a nucleic acid encoding an antibody of the disclosure; and (b) a cryoprotectant. In certain embodiments, the cryoprotectant comprises glycerol or DMSO. In certain embodiments, the master cell bank is contained in a suitable vial or container able to withstand freezing by liquid nitrogen.

Also described herein are methods of making an antibody described herein. Such methods comprise incubating a cell or cell-line comprising a nucleic acid encoding the antibody in a cell culture medium under conditions sufficient to allow for expression and secretion of the antibody, and further harvesting the antibody from the cell culture medium. The harvesting can further comprise one or more purification steps to remove live cells, cellular debris, non-antibody proteins or polypeptides, undesired salts, buffers, and medium components. In certain embodiments, the additional purification step(s) include centrifugation, ultracentrifugation, protein A, protein G, protein A/G, or protein L purification, and/or ion exchange chromatography.

“Treat,” “treatment,” or “treating,” as used herein refers to, e.g., a deliberate intervention to a physiological disease state resulting in the reduction in severity of a disease or condition; the reduction in the duration of a condition course; the amelioration or elimination of one or more symptoms associated with a disease or condition; or the provision of beneficial effects to a subject with a disease or condition. Treatment does not require curing the underlying disease or condition.

A “therapeutically effective amount,” “effective dose,” “effective amount,” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, “pharmaceutically acceptable” with reference to a carrier” “excipient” or “diluent” includes any and all solvents, dispersion media, coatings, antibacterial and antifungal agents, isotonic and absorption delaying agents, and the like that are physiologically compatible. In some aspects, the carrier is suitable for intravenous, intramuscular, subcutaneous, parenteral, spinal or epidermal administration (e.g., by injection or infusion). Depending on the route of administration, the active compound, i.e., antibody, can be coated in a material to protect the compound from the action of acids and other natural conditions that can inactivate the compound.

The pharmaceutical compounds described herein can include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66: 1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

As used herein, treatment or treating generally include and/or refer to a pharmaceutical or other intervention regimen used for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made. Skilled artisans will recognize that given a population of potential individuals for treatment not all will respond or respond equally to the treatment. Such individuals are considered treated.

In some embodiments, treating misfolded λ light chains and/or treating λ light chain amyloid deposits or treating λ light chain aggregates can be used interchangeably herein. In some embodiments, treating misfolded λ light chains includes treating a disorder characterized by misfolded λ light chains. In some embodiments, treating misfolded λ light chains comprises decreasing and/or reducing and/or inhibiting λ light chain aggregates (e.g., by measuring freely circulating light chains) or a symptom thereof. In some embodiments, treating misfolded λ light chains comprises promoting the clearance of treating misfolded λ light chains by monocytic cells and/or phagocytotic cells (e.g., by measuring freely circulating light chains).

As used herein, monocyte or monocyte cell refers and/or encompasses to a type of leukocyte, or white blood cell. Monocytes are the largest class of leukocyte and can differentiate into, e.g., macrophages. As a part of the vertebrate innate immune system monocytes also influence the process of adaptive immunity. Monocytes can generally be classified three types of monocytes in human blood: 1) The classical monocyte may be characterized by high level expression of the CD14 cell surface receptor (CD14⁺⁺ CD16⁻ monocyte); 2) The non-classical monocyte shows low level expression of CD14 and additional co-expression of the CD16 receptor (CD14⁺CD16⁺⁺ monocyte); and 3) The intermediate monocyte with high level expression of CD14 and low level expression of CD16 (CD14⁺⁺CD16⁺ monocytes).

As used herein, a macrophage generally refers to and/or encompasses a type of white blood cell of the immune system, that in a process called phagocytosis, engulfs and digests cellular debris, foreign substances, microbes, cancer cells, and anything else that does not have the type of proteins specific to healthy body cells on its surface. Beyond increasing inflammation and stimulating the immune system, macrophages can also play an important anti-inflammatory role and can decrease immune reactions through the release of cytokines

As used herein, a phagocytes generally refers to and/or encompasses a cell that is capable of phagocytosis and include both professional and non-professional phagocytes.

As used herein, amyloidosis generally refers to and/or encompasses a disease or condition characterized by the misfolding of free immunoglobulin light chains (LC) (e.g., lambda LCs) which can deposit as light chain amyloid (AL) in the heart, kidneys, or liver. The condition is typically caused by an underlying plasma cell dyscrasia that results in plasma cells secreting a large excess of free immunoglobulin kappa (κ) and/or lambda (λ) light chains into circulation. In certain instances, the immunoglobulin kappa (κ) and/or lambda (λ) light chains in circulation are misfolded, prone to aggregation and/or forming amyloid deposits, or aggregated Primary amyloidosis or AL amyloidosis generally refer to and/or encompass the most common type of systemic amyloidosis, with an incidence of 9-14 people per million per year in the Western world.

As used herein, plasma cell dyscrasia generally refers to and/or encompasses a proliferative plasma cell disorder under which a single, aberrant plasma cell clone (a terminally differentiated B cell) undergoes unregulated expansion and produces above-normal amounts of light chain protein (e.g., misfolded LC protein). Plasma cell dyscrasias can be classified as either benign, as seen in monoclonal gammopathies of unknown significance (MGUS), or malignant, as in multiple myeloma (MM) and plasma cell leukemia (PCL). Regardless of the type of dyscrasia involved, these secreted light chains can undergo misfolding events that render them prone to aggregate and accumulate as amyloid in vital organs including the heart, kidneys and nervous system. Such infiltration can result in nephrotic syndrome, hepatomegaly, peripheral neuropathy, restrictive cardiomyopathy, and ultimately death.

As used herein, the words comprising (and any form of comprising, such as comprise and comprises), having (and any form of having, such as have and has), including (and any form of including, such as include and includes) or containing (and any form of containing, such as contain and contains), are inclusive or open-ended and do not exclude additional, unrecited elements or process steps. As also used herein, in any instance or embodiment described herein, comprising may be replaced with consisting essentially of and/or consisting of. used herein, in any instance or embodiment described herein, comprises may be replaced with consists essentially of and/or consists of.

As used herein, the term about in the context of a given value or range includes and/or refers to a value or range that is within 20%, within 10%, and/or within 5% of the given value or range.

As used herein, the term and/or is to be taken as specific disclosure of each of the two specified features or components with or without the other. For example, A and/or B is to be taken as specific disclosure of each of (i) A, (ii) B and (iii) A and B, just as if each were set out individually herein.

### EXAMPLES

The following illustrative examples are representative of embodiments of compositions and methods described herein and are not meant to be limiting in any way.

### Example 1—Anti-Human λ Light Chain Antibodies

The problem solved, at least in part, was to create conformation-specific antibodies that bind the toxic light chain deposited in organs or their misfolded precursors in circulation, while leaving the well-folded, circulating light chains untouched. Solving this problem provides a solution to directly addressing the disease-causing species with high potency while avoiding interactions with the often-abundant, non-pathological folded, free light chains that could otherwise function as a biological sink during therapy or lead to unwanted immune side effects. Without being bound by theory binding of the antibody to the misfolded species targets them for removal via an antibody-dependent cellular phagocytosis (ADCP) mechanism.

Light chains are ˜214 amino acid proteins consisting of a variable domain (V_{L}, AA1-108) and a constant domain (C_{L}, AA109-214). The variable domain contains hypervariable regions which consists of different amino acid sequences in every patient, whereas the constant domain shares sequence identity in all patients with the same isotype (κ or λ). It is thought that in each patient, their unique light chain's variable domain sequence results in the protein's unique properties such as amyloidogenic propensity or amyloid structure, and much of the research efforts have been made to target this domain.

There are currently two competitor products aiming to use an antibody-based amyloid depletion mechanism to treat AL amyloidosis: Caelum CAEL-101 (ongoing Phase III) and NEOD001 (Phase III, failed). Both companies have used the strategy of creating a single antibody against the variable domain for the treatment of both κ- and λ-AL amyloidosis patient populations. While these antibodies purported to be effective for all patients (κ and λ), it was evident in both preclinical and clinical studies that the target engagement of these antibodies was inconsistent between individuals as demonstrated by immunohistochemistry and clinical response measurements. This inconsistency may be attributed to the epitope CAEL-101 and NEOD001. NEOD001 was an antibody originally designed to target serum amyloid A but was found to have cross-reactivity to light chains via interactions with a two-amino-acid epitope on the variable domain of the light chain protein. CAEL-101 was raised against an epitope in a κ AL patient. Recent cryo-EM structures of λ light chain amyloid extracted from patients showed that the epitopes targeted by CAEL-101 and NEOD001 may be buried in the core of the amyloid fiber (e.g., inaccessible to antibodies) in a patient's light chain, but accessible in another. This can lead to significant differences in the binding affinity and clinical efficacy between individuals.

The antibody design approach for generating anti-human λ light chain antibodies the was to identify a misfolding-specific epitope on the invariable constant domain, which will lead to antibodies that bind all variants of the same isotype with similar affinity. Laser capture microdissection mass spectrometry (LC-MS) studies to showed that the constant domain of light chains (not just the variable domain) is co-deposited in the affected tissues as full-length light chains or constant domain fragments, making them potential targets for antibody therapy utilizing the amyloid depletion mechanism. Accordingly, κ and λ light chains can also be considered two distinct targets.

The anti-human λ light chain antibodies described herein were generated to bind a misfolding-specific epitope, NKYAASSYLSL (SEQ ID NO: 51), on the λ light chain protein. To generate misfolding-specific antibodies, mice were immunized using an immunogen displaying the misfolding-specific epitope and subsequently harvested splenocytes and generated murine hybridoma cells using established methods. Thirteen clones were identified and purified from mouse ascites fluid for subsequent screening of desirable misfolding-specific properties.

### Antibody Binding to Denatured Light Chains

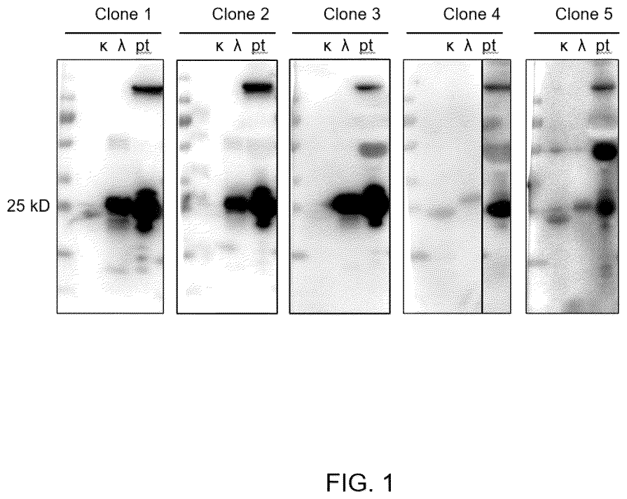

Initially, the ability of each of the thirteen clones to bind unfolded λ light chains was tested using western blotting to determine whether the antibodies can recognize our target epitope in the context of full-length unfolded λ light chains. λ light chains pooled from sera of multiple myeloma patients were used in the binding assay. For comparison, unfolded pooled κ immunoglobulin light chain was included, as well as whole serum from a patient with a confirmed diagnosis of λ light chain (AL) amyloidosis. FIG. 1 shows data demonstrating that five of the candidate antibodies successfully detected the denatured (SDS-unfolded) λ light chain protein, as well as one or more bands in the patient serum sample. The clones (Clones 1-5) all exhibited strong binding to denatured light chains. Clones 4-5 also appeared to cross-react with denatured κ light chains. Not all thirteen clones demonstrated binding to unfolded λ light chains. For the clones identified:

Clone 1 comprises CDRs identified/derived from a heavy chain variable domain comprising SEQ ID NO: 1, and a light chain variable domain comprising SEQ ID NO: 2.

Clone 2 comprises CDRs identified/derived from a heavy chain variable domain comprising SEQ ID NO: 11, and a light chain variable domain comprising SEQ ID NO: 12.

Clone 3 comprises CDRs identified/derived from a heavy chain variable domain comprising SEQ ID NO: 21, and a light chain variable domain comprising SEQ ID NO: 22.

Clone 4 comprises CDRs identified/derived from a heavy chain variable domain comprising SEQ ID NO: 31, and a light chain variable domain comprising SEQ ID NO: 32.

Clone 5 comprises CDRs identified/derived from a heavy chain variable domain comprising SEQ ID NO: 41, and a light chain variable domain comprising SEQ ID NO: 42.

### Preferential Binding to Misfolded Over Native Conformation

Of the five denatured light chain binders, whether these candidate monoclonal antibodies can distinguish and bind specifically to non-native λ light chain protein, and not to native (well-folded) form was addressed. As a representative λ light chain clone, Wil, a long-studied λ light chain variant first identified and sequenced in an individual with light chain amyloidosis was selected (Pokkuluri et al. 1999). Full-length, native Wil was produced by recombinant expression in vitro using established methods. Wil forms stable, native-like light chain dimers when expressed and purified under these conditions. Soluble, misfolded/unfolded Wil was then generated by treating the native protein with urea and reducing agents, followed by alkylation to permanently disrupt disulfide bond formation.

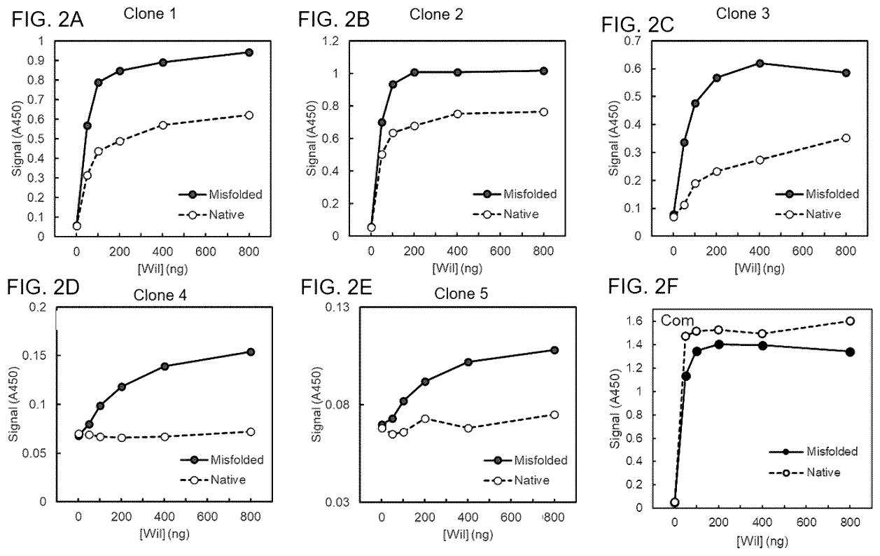

Indirect ELISA assays were performed to compare the binding of each antibody clone to native Wil versus misfolded/unfolded Wil. 96-well immunoassay plates were coated with a range of amounts of native or misfolded/unfolded Wil (from 0 to 800 ng of protein per well), and each well was probed with our candidate antibodies or a commercial control. Bound antibodies were detected using an HRP-conjugated secondary antibody and standard TMB chemistry, as exemplified by FIG. 2A-F. As demonstrated by FIG. 2A-B, Clone 1 and Clone 2 showed a high overall signal, respectively, but also substantial binding to native Wil (though less than to misfolded/unfolded Wil). As demonstrated by FIG. 2C, Clone 3 showed a slightly weaker signal, but slightly better discrimination between native and misfolded/unfolded Wil. As demonstrated by FIG. 2D-E, Clone 4 (FIG. 2D) and Clone 5 (FIG. 2E) exhibited the weakest overall signal, but also high specificity for misfolded Wil in this assay format. All five clones exhibited the ability to distinguish between misfolded/unfolded and native λ light chains to some degree, in contrast to the pan-specific commercial antibody shown in FIG. 2F.

### Preferential Binding to Aggregated/Amyloid Light Chains Over Native Conformation

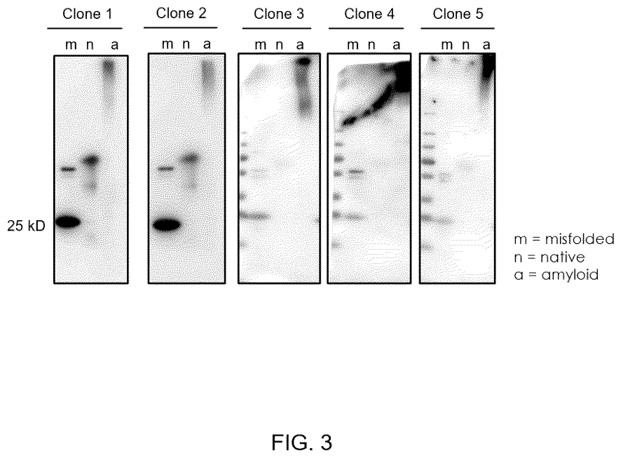

5 clones were examined for the ability to bind to aggregated forms (amyloid, disease-causing) of λ light chain using a native Western blot, as exemplified in FIG. 3. Formation of Wil aggregates were induced and amyloid through extended shaking at elevated temperatures using previously established methods and verified the formation of amyloid using the amyloid-sensitive dye, Thioflavin T. Native Western blot under ‘gentle’, low-detergent conditions designed to preserve the folding of protein samples were performed and compared the binding of our mAbs to native dimeric Wil; misfolded/unfolded Wil; and aggregated/amyloid Wil.

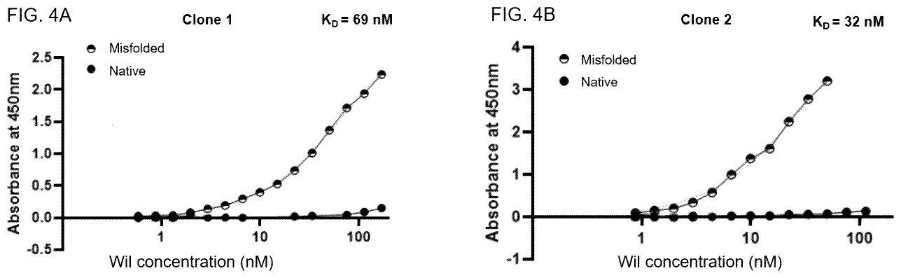

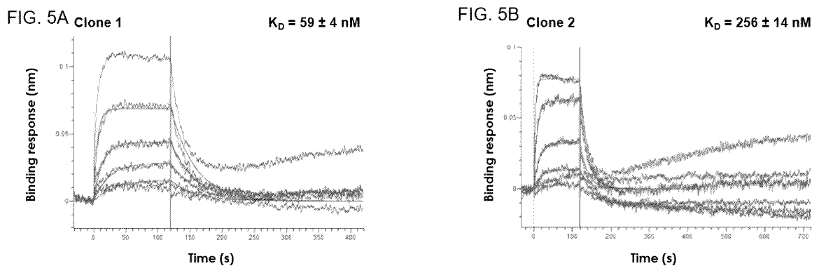

All the candidate mAbs detected both misfolded/unfolded and amyloid/aggregated Wil. But clones 1 and 2 exhibited the highest signal for the misfolded form of Wil. Although staining of the native bands was present, the signal was considerably reduced. It was suspected that the Western blotting and indirect ELISA methods, which immobilizes the ‘native’ Wil protein to a surface, can lead to false-positive binding to the native protein by inducing some degree of misfolding, which would not be detected by the clones that bound weakly (clones 3-5). As such, a capture format ELISA was used to interrogate the binding behavior of clones 1 and 2. In this format, a goat anti-mouse Fc capture antibody was coated into the ELISA wells, and clones 1 or 2 are captured by the antibody with their Fabs aligned to the appropriate orientation. Then, native, or misfolded forms of biotinylated Wil is added to these wells and binding is measured by streptavidin. As shown in FIGS. 4A and 4B, under these native conditions where Wil is in the solution phase, we observed minimal binding to native Wil and preferential binding to the misfolded form in both clones 1 and 2, suggesting conformation specificity. Both clone 1 (FIG. 5A) and clone 2 (FIG. 5B) had their affinities measured by bio-layer interferometry (BLI) indicating K_{D} of 59 nM and 256 nM respectively. Based on the above data, clone 1 was selected as the lead candidate for subsequent characterization (henceforth referred to as muLX-97).

In Vitro Characterization of muLX-97

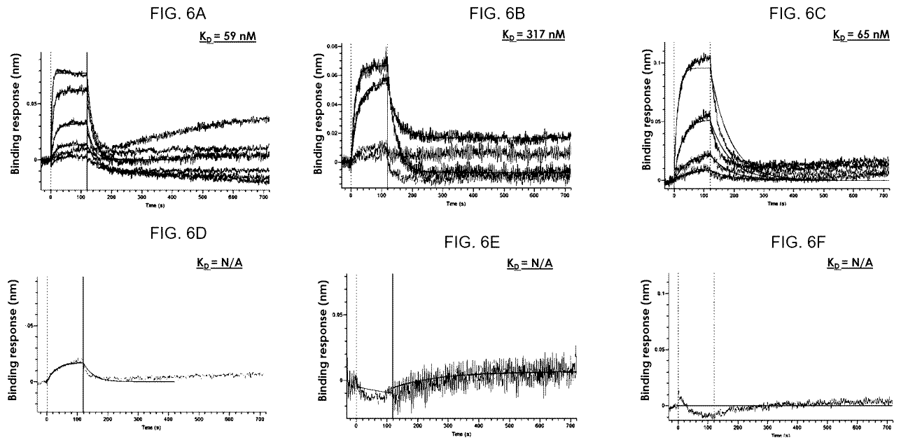

The binding affinity of muLX-97 to multiple λ light chain variants in misfolded and native conformations was then measured. In addition to Wil, two light chain variants sequenced from cardiac amyloid deposits in light chain amyloidosis patients were selected. See Oberti L et al. Concurrent structural and biophysical traits link with immunoglobulin light chains amyloid propensity. Sci Rep. 2017 Dec. 1; 7(1):16809 and Baur J et al. Identification of AL proteins from 10λ-AL amyloidosis patients by mass spectrometry extracted from abdominal fat and heart tissue. Amyloid. 2023 March; 30(1) 27-37. Again BLI was used to monitor the association and dissociation of misfolded and native forms of Wil (FIGS. 6A and 6D), H3 (FIGS. 6B and 6E) and FOR102 (FIGS. 6C and 6F) when binding to muLX-97. muLX-97 had binding affinities to misfolded Wil, H3, and FOR102 of 59, 317, and 65 nM respectively. In native conformations of all variants, only weak binding (limited BLI signal, at very high concentration of 1000 nM) was observed. This limited association may be due to the presence of small amounts of misfolded Wil contaminating the ‘native’ sample. Together, this demonstrates that the conformation specificity of muLX-97 can be generalized for all λ light chain variants.

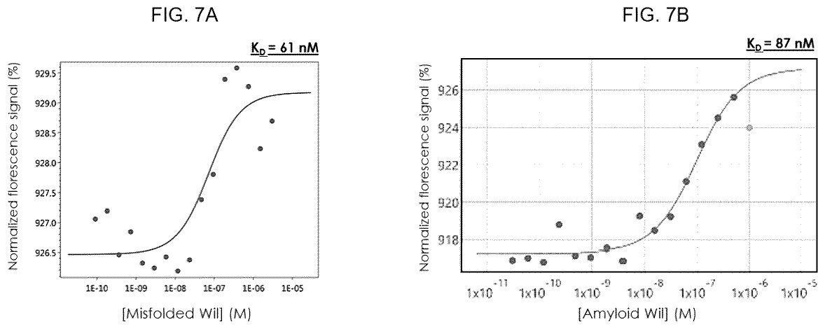

Next, the binding affinity of muLX-97 to amyloid forms of λ light chains was determined. A true affinity to aggregates could not be measured using previously described methods where either the antibody or target is immobilized, so microscale thermophoresis (MST) was used. In this method a small spot in a capillary containing fluorescently labeled antibody in the presence or absence of target is irradiated with an infrared laser. The local heating causes migration of protein out of the heated spot and the rate of migration change (due to the antibody binding to a ligand) can be used to measure the affinity. Using the NanoTemper Monolith, binding of muLX-97 to misfolded and amyloid forms of Wil was quantified, with results shown in FIG. 7. As shown in FIG. 7A the K_{D} to misfolded Wil was 61 nM, which matches the results from ELISA (FIG. 4A) and BLI (FIG. 6A), while FIG. 7B shows the affinity for amyloid Wil was 87 nM. These results support the notion that muLX-97 binds both the misfolded and amyloid forms of λ light chains with high specificity. The binding affinities of λ AL variants determined using various methods are summarized in Table 1.

TABLE 1
Binding affinities (K_{D}) of muLX-97 (clone 1) to various
λ light chain variants and conformations assessed
by various biophysical and immunoassays.

K_{D} (nM)

| Variant | Native | Misfolded | Amyloid |
|---|---|---|---|
| Wil | N/A (ELISA) | 69 (ELISA) | 87 (MST) |
| | N/A (BLI) | 59 (BLI) | |
| | | 61 (MST) | |
| H3 | N/A (BLI) | 317 (BLI) | |
| FOR102 | N/A (BLI) | 65 (BLI) | |

### Example 2—Anti-Human λ Light Chain Antibodies Bind to Human Tissue Amyloid

Ex Vivo Validation of muLX-97 Target Engagement

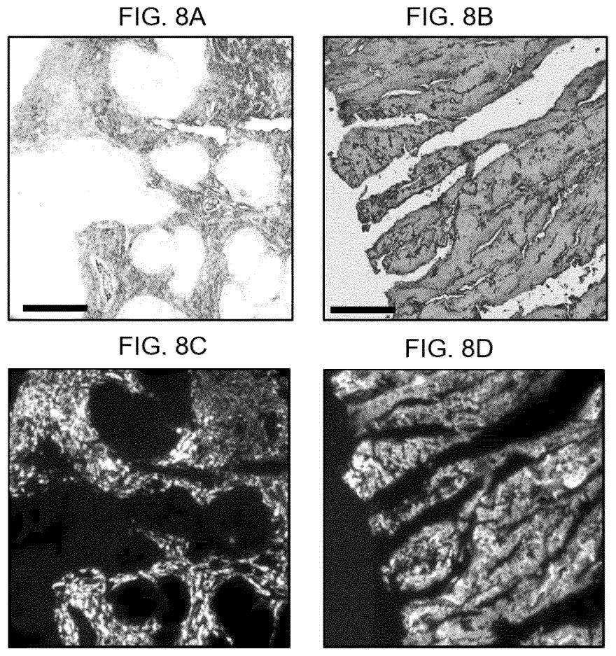

Next assessed was whether muLX-97 can engage disease-relevant targets in biopsy confirmed λ patient tissue. IHC+Congo Red co-staining of several cases of λ light chain amyloidosis patients was performed. Congo red is an amyloid-specific dye that can also be visualized by fluorescence microscopy. 2 representative cases are shown as examples. In the gastrointestinal case, the IHC staining pattern (FIG. 8A; dark staining) and the Congo red fluorescence pattern (FIG. 8C; white on dark background) and match closely in distribution and morphology. Similarly, this same co-staining is demonstrated in a soft tissue case (FIGS. 8B and 8D). The co-staining results show that muLX-97 stains morphologies consistent with amyloid and is thus amyloid-specific in biopsy confirmed cases of λ AL amyloidosis.

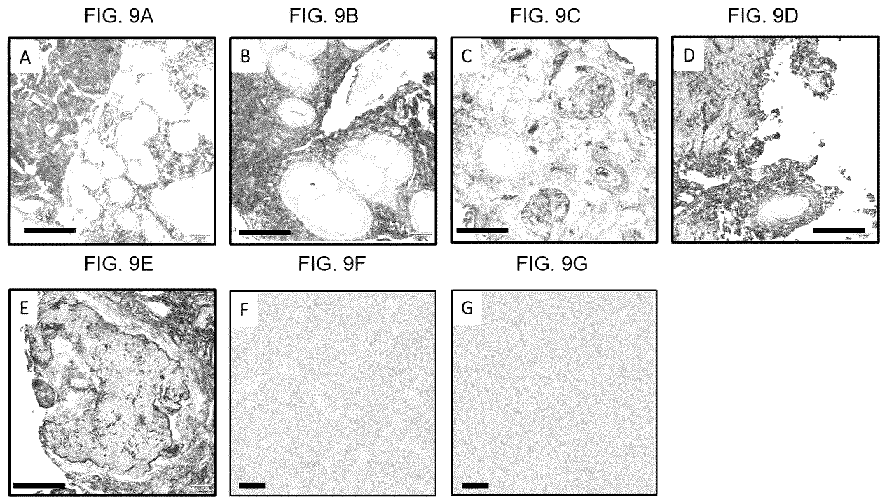

Positive staining in 5 cases of biopsy-confirmed λAL was also shown in gastrointestinal (FIGS. 9A and 9B), renal (FIG. 9C), and soft tissues (FIGS. 9D and 9E), while no staining was observed in pancreatic (FIG. 9F) or cardiac (FIG. 9G) tissues of patients with islet amyloid polypeptide amyloidosis or transthyretin amyloidosis, respectively. These results demonstrate that the disease-specific epitope described herein is likely a universal target present in all cases of λAL amyloidosis, and that an antibody raised against said epitope, such as muLX-97, is specific and targeted, with very low risk of cross-reactivity to other tissues.

### Example 3—Anti-Human λ Light Chain Antibodies Elicit ADCP Antibody-Dependent Phagocytosis of λAL Amyloid

After confirming binding affinity and target engagement of muLX-97, the ability of muLX-97 to elicit an immune response and lead to immune-mediated clearance of amyloid via antibody-mediate cellular phagocytosis (ADCP) was tested in a cellular model. In this model, amyloid Wil is conjugated to a pH-sensitive dye, pH-sensitive-rhodamine (pHrodo) and incubated with cultured macrophage cells and varying concentrations of muLX-97 or isotype control antibodies. If phagocytic uptake occurs, the amyloid will be shuttled into the lysosomes, where the decrease in pH results in fluorescence of the phagocytosing cell.

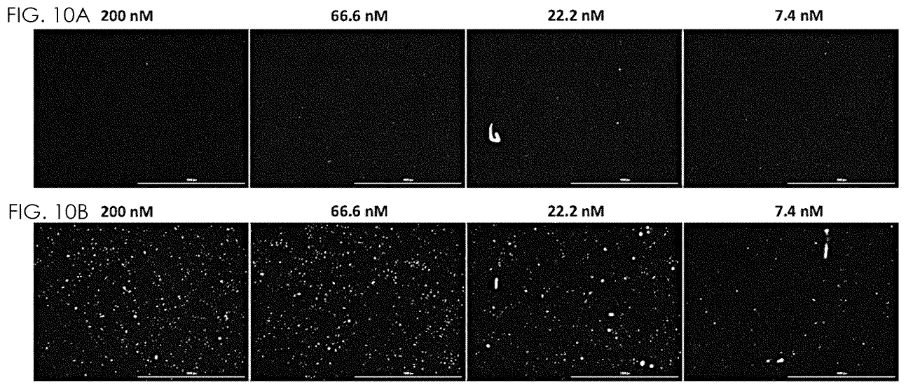

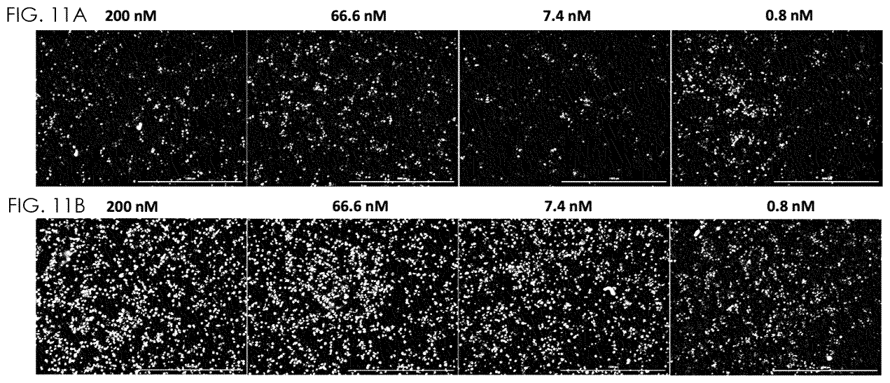

The ADCP model systems (RAW 264.7 and J774.1 cell lines) were used to assess the ability of muLX-97 to induce ADCP in mouse macrophages. 40,000 cells were incubated in 2 μM of pHrodo-labeled Wil amyloid and 0.1-200 nM of muLX-97 or isotype control antibody. In RAW264.7 murine macrophages, a significant increase in the proportion of pHrodo-positive cells incubated with muLX-97 (FIG. 10B) compared to the isotype antibody (FIG. 10A) at antibody concentrations as low as 7.4 nM was seen. In J744.1 cells, the phagocytosis effect was much more pronounced (up to 80% of cells were pHrodo-positive), and the enhancement of phagocytosis in muLX-97-treated cells (FIG. 11B) can be seen at 7.4 nM antibody concentration compared to those treated with isotype control (FIG. 11A).

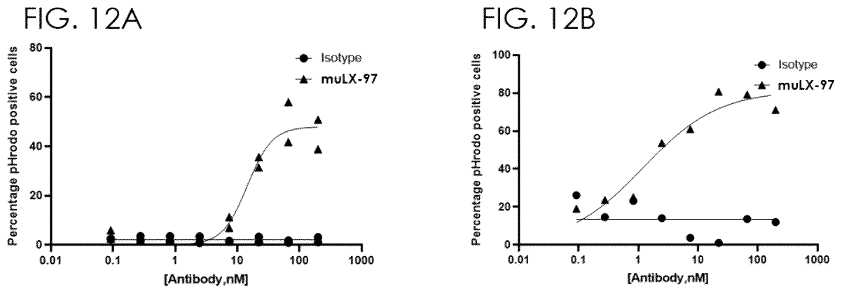

Quantifying the percent of pHrodo-positive cells as a function of antibody concentration in these 2 model systems show that muLX-97 can induce phagocytic uptake of λAL amyloid with an EC₅₀ of ˜20 nM and maximum signal of 50% in RAW 264.7 cells (FIG. 12A) and an EC₅₀ of ˜7 nM and maximum signal of 80% in J774.1 cells (FIG. 12B). Taken together, this cell model shows that muLX-97 significantly enhances macrophage-mediated clearance of λAL amyloid upon binding in a concentration-dependent manner, and that the potency of this effect occurs at substoichiometric concentrations of antibody.

While preferred embodiments of the present within a composition disclosure have been shown and described herein, it will be obvious to those skilled in the art that such embodiments are provided by way of example only. Numerous variations, changes, and substitutions will now occur to those skilled in the art without departing from the instant disclosure. It should be understood that various alternatives to the embodiments described herein may be employed in practicing the invention. It is intended that the following claims define the scope of the embodiments disclosed herein, and that methods and structures within the scope of these claims and their equivalents be covered thereby.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

| SEQ ID | | ANNOTATION |
|---|---|---|
| NO | SEQUENCE | |
| 1 | DVKLVESGGGLVKLGGSLKLSCAASGFTFS | CLONE- |
| | SYYMSWVRQTPEKRLELVAAINSNGGNTYY | 1 HV |
| | PDTVKGRFTISRDNAKNTLYLQMSSLKSED | |
| | TALYYCARLPFDYWGQGTTLTVSS | |
| 2 | DVVMTQTPLTLSVTIGQPASISCKSSQSLL | CLONE- |
| | DSDGKTYLNWLLQRPGQSPNRLIYLVSKLD | 1 LV |
| | SGVPDRFTGSGSGTDFTLKITRVEAEDLGV | |
| | YYCWQGTHFPHTFGAGTKLELK | |
| 3 | SYYMS | CLONE- |
| | | 1 H-CDR1 |
| 4 | AINSNGGNTYYPDTVKG | CLONE- |
| | | 1 H-CDR2 |
| 5 | LPFDY | CLONE- |
| | | 1 H-CDR3 |
| 6 | KSSQSLLDSDGKTYLN | CLONE- |
| | | 1 L-CDR1 |
| 7 | LVSKLDS | CLONE- |
| | | 1 L-CDR2 |
| 8 | WQGTHFPHT | CLONE- |
| | | 1 L-CDR3 |
| 9 | GACGTGAAGCTCGTGGAGTCTGGGGGAGGC | CLONE- |
| | TTAGTGAAGCTTGGAGGGTCCCTTAAACTC | 1 HV |
| | TCCTGTGCAGCCTCTGGATTCACTTTCAGT | |
| | AGCTATTACATGTCTTGGGTTCGCCAGACT | |
| | CCAGAGAAGAGGCTGGAGTTGGTCGCAGCC | |
| | ATTAATAGTAATGGTGGTAACACCTACTAT | |
| | CCAGACACTGTGAAGGGCCGATTCACCATC | |
| | TCCAGAGACAATGCCAAGAACACCCTGTAC | |
| | CTGCAAATGAGCAGTCTGAAGTCTGAGGAC | |
| | ACAGCCTTGTATTACTGTGCAAGACTCCCT | |
| | TTTGACTACTGGGGCCAAGGCACCACTCTC | |
| | ACAGTCTCCTCA | |
| 10 | GATGTTGTGATGACCCAGACTCCACTCACT | CLONE- |
| | TTGTCGGTTACCATTGGACAACCAGCCTCC | 1 LV |
| | ATCTCTTGCAAGTCAAGTCAGAGCCTCTTA | |
| | GATAGTGATGGAAAGACATATTTGAATTGG | |
| | TTGTTACAGAGGCCAGGCCAGTCTCCAAAT | |
| | CGCCTAATCTATCTGGTGTCTAAACTGGAC | |
| | TCTGGAGTCCCTGACAGGTTCACTGGCAGT | |
| | GGATCAGGGACAGATTTCACACTGAAAATC | |
| | ACCAGAGTGGAGGCTGAGGATTTGGGAGTT | |
| | TATTATTGCTGGCAAGGTACACATTTTCCT | |
| | CACACGTTCGGTGCTGGGACCAAGCTGGAG | |
| | CTGAAA | |
| 11 | DVKLVESGGGLVKLGGSLKLSCAASGFTFS | CLONE- |
| | NYYMSWVRQTPEKRLELVAAININGGTTYY | 2 HV |
| | PDTVKGRFTISRDNAKNTLYLQVSSLKSED | |
| | TALYYCARLPWDYWGQGTTLTVSS | |
| 12 | DVVMTQTPLTLSVTIGQPASISCKSSQSLL | CLONE- |
| | DSDGKTYLNWLLQRPGQSPKRLIYLVSKLD | 2 LV |
| | SGVPDRFTGSGSGTDFTLKISRVEAEDLGV | |
| | YYCWQGTHFPHTFGGGTKLEIK | |
| 13 | NYYMS | CLONE- |
| | | 2_H-CDR1 |
| 14 | AININGGTTYYPDTVKG | CLONE- |
| | | 2_H-CDR2 |
| 15 | LPWDY | CLONE- |
| | | 2 H-CDR3 |
| 16 | KSSQSLLDSDGKTYLN | CLONE- |
| | | 2 L-CDR1 |
| 17 | LVSKLDS | CLONE- |
| | | 2 L-CDR2 |
| 18 | WQGTHFPHT | CLONE- |
| | | 2 L-CDR3 |
| 19 | GACGTGAAGCTCGTGGAGTCTGGGGGAGGC | CLONE- |
| | TTAGTGAAGCTTGGAGGGTCCCTGAAACTC | 2 HV |
| | TCCTGTGCAGCCTCTGGATTCACTTTCAGT | |
| | AACTATTACATGTCTTGGGTTCGCCAGACT | |
| | CCAGAGAAGAGGCTGGAGTTGGTCGCAGCC | |
| | ATTAATATTAATGGTGGTACCACCTACTAT | |
| | CCAGACACTGTGAAGGGCCGATTCACCATC | |
| | TCCAGAGACAATGCCAAGAACACCCTGTAC | |
| | CTGCAAGTGAGCAGTCTGAAGTCTGAGGAC | |
| | ACAGCCTTGTATTACTGTGCAAGACTTCCC | |
| | TGGGACTACTGGGGCCAAGGCACCACTCTC | |
| | ACAGTCTCCTCA | |
| 20 | GATGTTGTGATGACCCAGACTCCACTCACT | CLONE- |
| | TTGTCGGTTACCATTGGACAACCAGCCTCC | 2 LV |
| | ATCTCTTGCAAGTCAAGTCAGAGCCTCTTA | |
| | GATAGTGATGGAAAGACATATTTGAATTGG | |
| | TTGTTACAGAGGCCAGGCCAGTCTCCAAAG | |
| | CGCCTAATCTATCTGGTGTCTAAACTGGAC | |
| | TCTGGAGTCCCTGACAGGTTCACTGGCAGT | |
| | GGATCAGGGACAGATTTCACACTGAAGATC | |
| | AGCAGAGTGGAGGCTGAGGATTTGGGAGTT | |
| | TATTATTGCTGGCAAGGTACACATTTTCCT | |
| | CACACGTTCGGAGGGGGGACCAAGCTGGAA | |
| | ATAAAA | |
| 21 | DVQLQESGPGLVKPSQSPSLTCTVTGYSIT | CLONE- |
| | SDYAWNWIRQFPGNKLEWMGDITYSGRTRY | 3 HV |
| | NPSLKSRISITRDTSKNQFFLQLKSVTTED | |
| | TGTYYCARSGSGAYWGQGTLLTVSA | |
| 22 | DIQMTQSSSYLSVSLGGRVTITCKASDHIN | CLONE- |
| | NWLAWYQQKPGNAPRLLISGATSLEIGVPS | 3 LV |
| | RFSGSGSGKDYTLSINSLQTEDVATYYCQQ | |
| | YWSTPFTFGSGTKLEIK | |
| 23 | SDYAWN | CLONE- |
| | | 3 H-CDR1 |
| 24 | DITYSGRTRYNPSLKS | CLONE- |
| | | 3 H-CDR2 |
| 25 | SGSGAY | CLONE- |
| | | 3 H-CDR3 |
| 26 | KASDHINNWLA | CLONE- |
| | | 3 L-CDR1 |
| 27 | GATSLEI | CLONE- |
| | | 3 L-CDR2 |
| 28 | QQYWSTPFT | CLONE- |
| | | 3 L-CDR3 |
| 29 | GATGTGCAGCTTCAGGAGTCGGGACCTGGC | CLONE- |
| | CTGGTGAAACCTTCTCAGTCTCCGTCCCTC | 3 HV |
| | ACCTGCACTGTCACTGGCTACTCAATCACC | |
| | AGTGATTATGCCTGGAACTGGATCCGGCAG | |
| | TTTCCAGGAAACAAACTGGAGTGGATGGGC | |
| | GACATAACTTACAGTGGTCGCACTCGGTAT | |
| | AATCCATCTCTCAAAAGTCGAATCTCTATC | |
| | ACTCGAGACACATCTAAGAACCAGTTCTTC | |
| | CTGCAGTTGAAATCTGTGACTACTGAAGAC | |
| | ACAGGCACATATTACTGTGCAAGAAGCGGC | |
| | TCCGGGGCTTACTGGGGCCAAGGGACTCTA | |
| | CTCACTGTCTCTGCA | |
| 30 | GACATCCAGATGACACAATCTTCATCCTAC | CLONE- |
| | TTGTCTGTATCTCTAGGAGGCAGAGTCACC | 3 LV |
| | ATTACTTGCAAGGCAAGTGACCACATTAAT | |
| | AATTGGTTAGCCTGGTATCAGCAGAAACCA | |
| | GGAAATGCTCCTAGGCTCTTAATATCTGGT | |
| | GCAACCAGTTTGGAAATTGGGGTTCCTTCA | |
| | AGATTCAGTGGCAGTGGATCTGGAAAGGAT | |
| | TACACTCTCAGCATTAACAGTCTTCAGACT | |
| | GAAGATGTTGCTACTTATTACTGTCAACAG | |
| | TATTGGAGTACTCCATTCACGTTCGGCTCG | |
| | GGGACAAAGTTGGAAATAAAA | |
| 31 | QIQLVQSGPELKKPGETVKISCKTSGFIST | CLONE- |
| | NFGMHWVKQAPGKGLKWMGWMNTYTGESTY | 4 HV |
| | ADEFKGRFAFSLETSANSVFVQINNLKNED | |
| | AARYFCAQISYDYDGYTMDFWGQGTSVTVS | |
| | S | |
| 32 | DVVLTQTPLSLPVRLGDQASISCRSGQSLV | CLONE- |
| | HRNGNTYFHWYLQKPGQSPNLLIYKVSIRF | 4 LV |
| | SGVPDRFSGSGSGTDFTLKISRVEAEDLGV | |
| | YFCSQSTHVPWTFGGGTKLEIK | |
| 33 | NFGMH | CLONE- |
| | | 4 H-CDR1 |
| 34 | WMNTYTGESTYADEFKG | CLONE- |
| | | 4 H-CDR2 |
| 35 | ISYDYDGYTMDF | CLONE- |
| | | 4 H-CDR3 |
| 36 | RSGQSLVHRNGNTYFH | CLONE- |
| | | 4 L-CDR1 |
| 37 | KVSIRFS | CLONE- |
| | | 4 L-CDR2 |
| 38 | SQSTHVPWT | CLONE- |
| | | 4 L-CDR3 |
| 39 | CAGATCCAGTTGGTGCAGTCTGGACCTGAG | CLONE- |
| | CTGAAGAAGCCTGGAGAGACAGTCAAGATC | 4 HV |
| | TCCTGCAAGACTTCTGGGTTTATCTCCACA | |
| | AATTTTGGAATGCACTGGGTCAAGCAGGCT | |
| | CCAGGAAAGGGTTTAAAGTGGATGGGCTGG | |
| | ATGAACACTTACACTGGAGAGTCAACATAT | |
| | GCTGATGAATTCAAGGGACGATTTGCCTTC | |
| | TCTTTGGAAACGTCTGCCAACTCTGTCTTT | |
| | GTGCAGATCAACAACCTCAAAAATGAGGAC | |
| | GCGGCCAGATATTTCTGTGCCCAGATCTCC | |
| | TATGATTACGACGGATATACTATGGACTTC | |
| | TGGGGTCAAGGAACCTCAGTCACCGTCTCC | |
| | TCA | |
| 40 | GATGTTGTGCTGACCCAAACTCCACTCTCC | CLONE- |
| | CTGCCTGTCCGTCTTGGAGATCAGGCCTCC | 4 LV |
| | ATCTCTTGCAGATCTGGTCAGAGCCTTGTA | |
| | CACAGAAATGGAAACACCTATTTTCATTGG | |
| | TACCTGCAGAAGCCAGGCCAGTCTCCAAAT | |
| | CTCCTGATCTACAAAGTATCCATCCGATTT | |
| | TCTGGGGTCCCAGACAGGTTCAGTGGCAGT | |
| | GGATCAGGGACAGATTTCACACTCAAGATC | |
| | AGCAGAGTGGAGGCTGAGGATCTGGGAGTT | |
| | TATTTCTGCTCTCAAAGTACACATGTTCCG | |
| | TGGACGTTCGGTGGAGGCACCAAGCTGGAA | |
| | ATCAAA | |
| 41 | QIQLVQSGPELKKPGETVKISCKTSGYFFT | CLONE- |
| | NFGMHWVKQAPGKGLKWMGWMNTYTGESTY | 5 HV |
| | ADDFKGRFAFSLETSASTVCLHINILKNED | |
| | TATYFCAQISYDYDGYTMDYWGQGTSVTVS | |
| | S | |
| 42 | DVVMTQTPLSLPVSLGDQASISCRSSQSLV | CLONE- |
| | HSNGNTYLHWYLQKPGQSPNLLIYKLSNRF | 5 LV |
| | SGVPDRFSGSGSGTDFTLKISRVEAEDLGV | |
| | YFCSQSTHVPWTFGGGTKLEIK | |
| 43 | NFGMH | CLONE- |
| | | 5 H-CDR1 |
| 44 | WMNTYTGESTYADDFKG | CLONE- |
| | | 5 H-CDR2 |
| 45 | ISYDYDGYTMDY | CLONE- |
| | | 5 H-CDR3 |
| 46 | RSSQSLVHSNGNTYLH | CLONE- |
| | | 5 L-CDR1 |
| 47 | KLSNRFS | CLONE- |
| | | 5_L-CDR2 |
| 48 | SQSTHVPWT | CLONE- |
| | | 5 L-CDR3 |
| 49 | CAGATCCAGTTGGTGCAGTCTGGACCTGAG | CLONE- |
| | CTGAAGAAGCCTGGAGAGACAGTCAAGATC | 5 HV |
| | TCCTGCAAGACTTCTGGGTATTTCTTCACA | |
| | AACTTTGGAATGCACTGGGTCAAGCAGGCT | |
| | CCAGGAAAGGGTTTAAAGTGGATGGGCTGG | |
| | ATGAACACCTACACTGGAGAGTCAACATAT | |
| | GCTGATGACTTCAAGGGACGGTTTGCCTTC | |
| | TCTTTGGAAACCTCTGCCAGCACTGTCTGT | |
| | TTGCATATCAACATCCTCAAAAATGAGGAC | |
| | ACGGCTACATATTTCTGTGCCCAGATCTCC | |
| | TATGATTACGACGGATATACTATGGACTAC | |
| | TGGGGTCAAGGAACCTCAGTCACCGTCTCC | |
| | TCA | |
| 50 | GATGTTGTGATGACCCAAACTCCACTCTCC | CLONE- |
| | CTGCCTGTCAGTCTTGGAGATCAAGCCTCC | 5 LV |
| | ATCTCTTGCAGATCTAGTCAGAGCCTTGTA | |
| | CACAGTAATGGAAACACCTATTTACATTGG | |
| | TACCTGCAGAAGCCAGGCCAGTCTCCAAAT | |
| | CTCCTGATCTACAAACTTTCCAACCGATTT | |
| | TCTGGGGTCCCAGACAGGTTCAGTGGCAGT | |
| | GGATCAGGGACAGATTTCACACTCAAGATC | |
| | AGCAGAGTGGAGGCTGAGGATCTGGGAGTT | |
| | TATTTCTGCTCTCAAAGTACACATGTTCCG | |
| | TGGACGTTCGGTGGAGGCACCAAGCTGGAA | |
| | ATCAAA | |
| 51 | NKYAASSYLSL | LCL |
| | | EPITOPE |
| 52 | ASTKGPSVFPLAPSSKSTSGGTAALGCLVK | HC |
| | DYFPEPVTVSWNSGALTSGVHTFPAVLQSS | |
| | GLYSLSSVVTVPSSSLGTQTYICNVNHKPS | |
| | NTKVDKKVEPKSCDKTHTCPPCPAPELLGG | |
| | PSVFLFPPKPKDTLMISRTPEVTCVVVDVS | |
| | HEDPEVKFNWYVDGVEVHNAKTKPREEQYN | |
| | STYRVVSVLTVLHQDWLNGKEYKCKVSNKA | |
| | LPAPIEKTISKAKGQPREPQVYTLPPSRDE | |
| | LTKNQVSLTCLVKGFYPSDIAVEWESNGQP | |
| | ENNYKTTPPVLDSDGSFFLYSKLTVDKSRW | |
| | QQGNVFSCSVMHEALHNHYTQKSLSLSPGK | |

1. An anti-human lambda light chain antibody or human lambda light chain binding fragment thereof comprising:
(a) a heavy chain complementarity determining region 1 (H-CDR1) comprising an amino acid sequence set forth in SEQ ID NO: 3;
(b) a heavy chain complementarity determining region 2 (H-CDR2) comprising an amino acid sequence set forth in SEQ ID NO: 4;
(c) a heavy chain complementarity determining region 3 (H-CDR3) comprising an amino acid sequence set forth in SEQ ID NO: 5;
(d) a light chain complementarity determining region 1 (L-CDR1) comprising an amino acid sequence set forth in SEQ ID NO: 6;
(e) a light chain complementarity determining region 2 (L-CDR2) comprising an amino acid sequence set forth in SEQ ID NO: 7; and/or
(f) a light chain complementarity determining region 3 (L-CDR3) comprising an amino acid sequence set forth in SEQ ID NO: 8.

2. The anti-human lambda light chain antibody or human lambda light chain binding fragment thereof of claim 1, comprising:
a heavy chain variable region comprising the H-CDR1, H-CDR2, and H-CDR3;
and/or
a light chain variable region comprising the L-CDR1, L-CDR2, and L-CDR3.

3. The anti-human lambda light chain antibody or human lambda light chain binding fragment thereof of claim 2, wherein:
the heavy chain variable region comprises an amino acid sequence having at least about 80% identity to SEQ ID NO: 1; and/or
the light chain variable region comprises an amino acid sequence having at least about 80% identity to SEQ ID NO: 2.

4. An anti-human lambda light chain antibody or human lambda light chain binding fragment thereof comprising:
(a) a heavy chain complementarity determining region 1 (H-CDR1) comprising an amino acid sequence set forth in SEQ ID NO: 13;
(b) a heavy chain complementarity determining region 2 (H-CDR2) comprising an amino acid sequence set forth in SEQ ID NO: 14;
(c) a heavy chain complementarity determining region 3 (H-CDR3) comprising an amino acid sequence set forth in SEQ ID NO: 15;
(d) a light chain complementarity determining region 1 (L-CDR1) comprising an amino acid sequence set forth in SEQ ID NO: 16;
(e) a light chain complementarity determining region 2 (L-CDR2) comprising an amino acid sequence set forth in SEQ ID NO: 17; and/or
(f) a light chain complementarity determining region 3 (L-CDR3) comprising an amino acid sequence set forth in SEQ ID NO: 18.

5. The anti-human lambda light chain antibody or human lambda light chain binding fragment thereof of claim 4, comprising:
a heavy chain variable region comprising the H-CDR1, H-CDR2, and H-CDR3;
and/or
a light chain variable region comprising the L-CDR1, L-CDR2, and L-CDR3.

6. The anti-human lambda light chain antibody or human lambda light chain binding fragment thereof of claim 5, wherein:
the heavy chain variable region comprises an amino acid sequence having at least about 80% identity to SEQ ID NO: 11; and/or
the light chain variable region comprises an amino acid sequence having at least about 80% identity to SEQ ID NO: 12.

7. An anti-human lambda light chain antibody or human lambda light chain binding fragment thereof comprising:
(a) a heavy chain complementarity determining region 1 (H-CDR1) comprising an amino acid sequence set forth in SEQ ID NO: 23;
(b) a heavy chain complementarity determining region 2 (H-CDR2) comprising an amino acid sequence set forth in SEQ ID NO:24;
(c) a heavy chain complementarity determining region 3 (H-CDR3) comprising an amino acid sequence set forth in SEQ ID NO: 25;
(d) a light chain complementarity determining region 1 (L-CDR1) comprising an amino acid sequence set forth in SEQ ID NO: 26;
(e) a light chain complementarity determining region 2 (L-CDR2) comprising an amino acid sequence set forth in SEQ ID NO: 27; and/or
(f) a light chain complementarity determining region 3 (L-CDR3) comprising an amino acid sequence set forth in SEQ ID NO: 28.

8. The anti-human lambda light chain antibody or human lambda light chain binding fragment thereof of claim 7, comprising:
a heavy chain variable region comprising the H-CDR1, H-CDR2, and H-CDR3;
and/or
a light chain variable region comprising the L-CDR1, L-CDR2, and L-CDR3.

9. The anti-human lambda light chain antibody or human lambda light chain binding fragment thereof of claim 8, wherein:
the heavy chain variable region comprises an amino acid sequence having at least about 80% identity to SEQ ID NO: 21; and/or
the light chain variable region comprises an amino acid sequence having at least about 80% identity to SEQ ID NO: 22.

10. An anti-human lambda light chain antibody or human lambda light chain binding fragment thereof comprising:
(a) a heavy chain complementarity determining region 1 (H-CDR1) comprising an amino acid sequence set forth in SEQ ID NO: 33;
(b) a heavy chain complementarity determining region 2 (H-CDR2) comprising an amino acid sequence set forth in SEQ ID NO: 34;
(c) a heavy chain complementarity determining region 3 (H-CDR3) comprising an amino acid sequence set forth in SEQ ID NO: 35;
(d) a light chain complementarity determining region 1 (L-CDR1) comprising an amino acid sequence set forth in SEQ ID NO: 36;
(e) a light chain complementarity determining region 2 (L-CDR2) comprising an amino acid sequence set forth in SEQ ID NO: 37; and/or
(f) a light chain complementarity determining region 3 (L-CDR3) comprising an amino acid sequence set forth in SEQ ID NO: 38.

11. The anti-human lambda light chain antibody or human lambda light chain binding fragment thereof of claim 10, comprising:
a heavy chain variable region comprising the H-CDR1, H-CDR2, and H-CDR3;
and/or
a light chain variable region comprising the L-CDR1, L-CDR2, and L-CDR3.

12. The anti-human lambda light chain antibody or human lambda light chain binding fragment thereof of claim 11, wherein:
the heavy chain variable region comprises an amino acid sequence having at least about 80% identity to SEQ ID NO: 31; and/or
the light chain variable region comprises an amino acid sequence having at least about 80% identity to SEQ ID NO: 32.

13. An anti-human lambda light chain antibody or human lambda light chain binding antibody fragment thereof comprising:
(a) a heavy chain complementarity determining region 1 (H-CDR1) comprising an amino acid sequence set forth in SEQ ID NO: 43;
(b) a heavy chain complementarity determining region 2 (H-CDR2) comprising an amino acid sequence set forth in SEQ ID NO:44;
(c) a heavy chain complementarity determining region 3 (H-CDR3) comprising an amino acid sequence set forth in SEQ ID NO: 45;
(d) a light chain complementarity determining region 1 (L-CDR1) comprising an amino acid sequence set forth in SEQ ID NO: 46;
(e) a light chain complementarity determining region 2 (L-CDR2) comprising an amino acid sequence set forth in SEQ ID NO: 47; and/or
(f) a light chain complementarity determining region 3 (L-CDR3) comprising an amino acid sequence set forth in SEQ ID NO: 48.

14. The anti-human lambda light chain antibody or human lambda light chain binding fragment thereof of claim 13, comprising:
a heavy chain variable region comprising the H-CDR1, H-CDR2, and H-CDR3;
and/or
a light chain variable region comprising the L-CDR1, L-CDR2, and L-CDR3.

15. The anti-human lambda light chain antibody or human lambda light chain binding fragment thereof of claim 14, wherein:
the heavy chain variable region comprises an amino acid sequence having at least about 80% identity to SEQ ID NO: 41; and/or
the light chain variable region comprises an amino acid sequence having at least about 80% identity to SEQ ID NO: 42.

16. An anti-human lambda light chain antibody or human lambda light chain antibody fragment thereof comprising:
a heavy chain variable region comprising an amino acid sequence having at least about 90%, 95%, 97%, 98%, or 99% identity to SEQ ID NO: 1; and/or
a light chain variable region comprising an amino acid sequence having at least about 90%, 95%, 97%, 98%, or 99% identity to SEQ ID NO: 2.

17. An anti-human lambda light chain antibody or human lambda light chain binding antibody fragment thereof comprising:
a heavy chain variable region comprising an amino acid sequence as set forth in SEQ ID NO: 1; and/or
a light chain variable region comprising an amino acid sequence as set forth in SEQ ID NO: 2.

18. An anti-human lambda light chain antibody or human lambda light chain binding fragment thereof comprising:
a heavy chain variable region comprising an amino acid sequence having at least about 90%, 95%, 97%, 98%, or 99% identity to SEQ ID NO: 11; and/or
a light chain variable region comprising an amino acid sequence having at least about 90%, 95%, 97%, 98%, or 99% identity to SEQ ID NO: 12.

19. An anti-human lambda light chain antibody or human lambda light chain binding fragment thereof comprising:
a heavy chain variable region comprising an amino acid sequence as set forth in SEQ ID NO: 11; and/or
a light chain variable region comprising an amino acid sequence as set forth in SEQ ID NO: 12.

20. An anti-human lambda light chain antibody or human lambda light chain binding fragment thereof comprising:
a heavy chain variable region comprising an amino acid sequence having at least about 90%, 95%, 97%, 98%, or 99% identity to SEQ ID NO: 21; and/or
a light chain variable region comprising an amino acid sequence having at least about 90%, 95%, 97%, 98%, or 99% identity to SEQ ID NO: 22.

21. An anti-human lambda light chain antibody or human lambda light chain binding fragment thereof comprising:
a heavy chain variable region comprising an amino acid sequence as set forth in SEQ ID NO: 21; and/or
a light chain variable region comprising an amino acid sequence as set forth in SEQ ID NO: 22.

22. An anti-human lambda light chain antibody or human lambda light chain binding fragment thereof comprising:
a heavy chain variable region comprising an amino acid sequence having at least about 90%, 95%, 97%, 98%, or 99% identity to SEQ ID NO: 31; and/or
a light chain variable region comprising an amino acid sequence having at least about 90%, 95%, 97%, 98%, or 99% identity to SEQ ID NO: 32.

23. An anti-human lambda light chain antibody or human lambda light chain binding fragment thereof comprising:
a heavy chain variable region comprising an amino acid sequence as set forth in SEQ ID NO: 31; and/or
a light chain variable region comprising an amino acid sequence as set forth in SEQ ID NO: 32.

24. An anti-human lambda light chain antibody or human lambda light chain binding fragment thereof comprising:
a heavy chain variable region comprising an amino acid sequence having at least about 90%, 95%, 97%, 98%, or 99% identity to SEQ ID NO: 41; and/or
a light chain variable region comprising an amino acid sequence having at least about 90%, 95%, 97%, 98%, or 99% identity to SEQ ID NO: 42.

25. An anti-human lambda light chain antibody or human lambda light chain binding fragment thereof comprising:
a heavy chain variable region comprising an amino acid sequence as set forth in SEQ ID NO: 41; and/or
a light chain variable region comprising an amino acid sequence as set forth in SEQ ID NO: 42.

26. The anti-human lambda light chain antibody or human lambda light chain binding fragment thereof of any one of claims 1 to 25, wherein anti-human λ light chain antibody is a monoclonal antibody.

27. The anti-human lambda light chain antibody or human lambda light chain binding fragment thereof of any one of claims 1 to 26, wherein the anti-human λ light chain antibody is a chimeric antibody, a humanized antibody, or human antibody.

28. The anti-human lambda light chain antibody or human lambda light chain binding fragment thereof of any one of claims 1 to 25, wherein the human λ light chain binding antibody fragment comprises a Fab, a Fab′, a F(ab′)₂, a scFv, a dsFv, a ds-scFv, a dimer, a minibody, or a diabody.

29. The anti-human lambda light chain antibody or human lambda light chain binding fragment thereof of any one of claims 1 to 25, wherein the anti-human lambda light chain antibody or human lambda light chain binding fragment thereof is an IgG antibody.

30. The anti-human lambda light chain antibody or human lambda light chain binding fragment thereof of claim 29, wherein the IgG antibody is an IgG1 isotype.

31. The anti-human lambda light chain antibody or human lambda light chain binding fragment thereof of claim 29, wherein the IgG antibody is an IgG4 isotype.

32. The anti-human lambda light chain antibody or human lambda light chain binding fragment thereof of any one of claims 1 to 31, wherein the anti-human lambda light chain antibody or human lambda light chain binding fragment thereof binds to an epitope comprising or consisting of SEQ ID NO: 51.

33. The method of claim 32, wherein binding to the epitope comprising SEQ ID NO 51 is measured by ELISA, surface plasmon resonance, bio-layer interferometry, or isothermal calorimetry.

34. The anti-human lambda light chain antibody or human lambda light chain binding fragment thereof of any one of claims 1 to 33, wherein the anti-human lambda light chain antibody or human lambda light chain binding fragment thereof is coupled to a label.

35. The anti-human lambda light chain antibody or human lambda light chain binding fragment thereof of claim 34, wherein the label comprises a chemical label, a biological label, a fluorescent label, a radioisotope label, or any combination thereof.

36. A pharmaceutical composition comprising the anti-human lambda light chain antibody or human lambda light chain binding fragment thereof of any one of claims 1 to 35, and a pharmaceutically acceptable excipient, diluent, or carrier.

37. A pharmaceutical composition comprising one or more of the anti-human lambda light chain antibody or human lambda light chain binding fragment thereof of any one of claims 1 to 35, and a pharmaceutically acceptable excipient, diluent, or carrier.

38. A nucleic acid encoding the anti-human lambda light chain antibody or human lambda light chain binding fragment thereof of any one of claims 1 to 33.

39. A host cell comprising a nucleic acid encoding the anti-human lambda light chain antibody or human lambda light chain binding fragment thereof of any one of claims 1 to 33.

40. A method of diagnosing amyloidosis in a sample, the method comprising contacting a biological sample obtained from a subject with the anti-human lambda light chain antibody or human lambda light chain binding fragment of any one of claims 1 to 35.

41. A method of detecting free light chain polypeptides in a sample, the method comprising contacting the biological sample with the anti-human lambda light chain antibody or human lambda light chain binding fragment thereof of any one of claims 1 to 35.

42. A method of detecting amyloid deposits in a sample, the method comprising contacting the biological sample with the anti-human lambda light chain antibody or human lambda light chain binding fragment thereof of any one of claims 1 to 35.

43. The method of any one of claims 40 to 42, wherein the sample is from blood, serum, plasma, solid tissue, or any combination thereof.

44. The method of any one of claims 40 to 42, wherein the sample is from a subject.

45. A method of promoting the clearance of a λ light chain aggregate by monocytic cells, the method comprising contacting the λ light chain aggregate with the anti-human lambda light chain antibody or human lambda light chain binding fragment thereof of any one of claims 1 to 35.

46. A method of increasing the clearance of a λ light chain aggregates, the method comprising contacting the λ light chain aggregate with the anti-human lambda light chain antibody or human lambda light chain binding fragment thereof of any one of claims 1 to 35.

47. A method of reducing lambda light chain aggregates in a subject, the method comprising administering the anti-human lambda light chain antibody or human lambda light chain binding fragment thereof of any one of claims 1 to 35 to the subject, thereby reducing the number of lambda light chain aggregates in the subject.

48. A method of treating a disease or condition characterized by lambda light chain aggregates, the method comprising administering the anti-human lambda light chain antibody or human lambda light chain binding fragment thereof of any one of claims 1 to 35 to the subject, thereby reducing the number of lambda light chain aggregates in the subject.

49. The method of any one of claims 47 or 48, wherein the subject is afflicted with a plasma cell disorder.

50. The method of claim 49, wherein the plasma cell disorder comprises plasma cell dyscrasias.

51. The method of claim 50, wherein the plasma cell dyscrasia is selected from a monoclonal gammopathy of unknown significance (MGUS), multiple myeloma (MM), plasma cell leukemia (PCL), and any combination thereof.

52. The method of any one of claims 47 or 48, wherein the subject is afflicted with systemic amyloidosis.

53. The method of any one of claims 47 or 48, wherein the subject is afflicted with AL amyloidosis.
